(19) United States
(12) Patent Application Publication
(10) Pub. No.: US 2005/0154230 A1
(43) Pub. Date: Jul. 14, 2005

(54) Urea derivatives

(75) Inventors: Takeshi Yura, Handa-shi (JP); Muneto Mogi, Nara-shi (JP); Yuka Ikegami, Kyoto-shi (JP); Tsutomu Masuda, Handa-shi (JP); Toshio Kokubo, Nara-shi (JP); Klaus Urbahns, Lund (SE); Nagahiro Yoshida, Soraku-gun (JP); Makiko Marumo, Nara-shi (JP); Masahiro Shiroo, Cambridge (GB); Masaomi Tajimi, Chita-shi (JP); Keisuke Takeshita, Takamatsu-shi (JP); Toshiya Moriwaki, Ikoma-shi (JP); Yasuhiro Tsukimi, Amagasaki-shi (JP)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/499,785

(22) PCT Filed: Dec. 13, 2002
(86) PCT No.: PCT/EP02/14216

(30) Foreign Application Priority Data
Dec. 26, 2001 (JP) 2001-395032
Dec. 26, 2001 (JP) 2001-395033

(51) Int. Cl. A61K 31/17; C07C 275/32

(52) U.S. Cl. 564/52; 514/598

(57) ABSTRACT

This invention relates to urea derivatives and salts thereof which is useful as an active ingredient of pharmaceutical preparations. The urea derivatives of the present invention has an excellent activity as VR1 antagonist and useful for the prophylaxis and treatment of diseases associated with VR1 activity, in particular for the treatment of urge urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

### DETAILED DESCRIPTION OF INVENTION

1. Technical Field

The present invention relates to an urea derivative, which is useful as an active ingredient of pharmaceutical preparations. The urea derivatives of the present invention have vanilloid receptor (VR1) antagonistic activity, and can be used for the prophylaxis and treatment of diseases associated with VR1 activity, in particular for the treatment of urge urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

2. Background Art

Vanilloid compounds are characterized by the presence of vanillyl group or a functionally equivalent group. Examples of several vanilloid compounds or vanilloid receptor modulators are vanillin (4-hydroxy-3-methoxy-benzaldehyde), guaiacol, (2-methoxy-phenol), zingerone (4-/4-hydroxy-3-methoxyphenyl/-2-butanon), eugenol (2-methoxy4-/2-propenyl/phenol), and capsaicin (8-methy-N-vanillyl-6-noneneamide).

Among others, capsaicin, the main pungent ingredient in “hot” chili peppers, is a specific neurotoxin that desensitizes C-fiber afferent neurons. Capsaicin interacts with vanilloid receptors (VR1), which are predominantly expressed in cell bodies of dorsal root ganglia (DRG) or nerve endings of afferent sensory fibers including C-fiber nerve endings [Tominaga M, Caterina M J, Malmberg A B, Rosen T A, Gilbert H, Skinner K, Raumann B E, Basbaum A I, Julius D: The cloned capsaicin receptor integrates multiple pain-producing stimuli. Neuron. 21: 531-543, 1998]. The VR1 receptor was recently cloned [Caterina M J, Schumacher M A, Tominaga M, Rosen T A, Levine J D, Julius D: Nature 389: 816-824, (1997)] and identified as a nonselective cation channel with six transmembrane domains that is structurally related to the TRP (transient receptor potential) channel family. Binding of capsaicin to VR1 allows sodium, calcium and possibly potassium ions to flow down their concentration gradients, causing initial depolarization and release of neurotransmitters from the nerve terminals. VR1 can therefore be viewed as a molecular integrator of chemical and physical stimuli that elicit neuronal signals in a pathological conditions or diseases.

There are abundant of direct or indirect evidence that shows the relation between VR1 activity and diseases such as pain, ischaemia, and inflammatory (e.g., WO 99/00115 and 00/50387). Further, it has been demonstrated that VR1 transduce reflex signals that are involved in the overactive bladder of patients who have damaged or abnormal spinal reflex pathways [De Groat W C: A neurologic basis for the overactive bladder. Urology 50 (6A Suppl): 36-52, 1997]. Desensitisation of the afferent nerves by depleting neurotransmitters using VR1 agonists such as capsaicin has been shown to give promising results in the treatment of bladder dysfunction associated with spinal cord injury and multiple sclerosis [(Maggi C A: Therapeutic potential of capsaicin-like molecules—Studies in animals and humans. Life Sciences 51: 1777-1781, 1992) and (DeRidder D; Chandiramani V; Dasgupta P; VanPoppel H; Baert L; Fowler C J: Intravesical capsaicin as a treatment for refractory detrusor hyperreflexia: A dual center study with long-term followup. J. Urol. 158: 2087-2092, 1997)].

It is anticipated that antagonism of the VR1 receptor would lead to the blockage of neurotransmitter release, resulting in prophylaxis and treatment of the condition and diseases associated with VR1 activity.

It is therefore expected that antagonists of the VR1 receptor can be used for prophylaxis and treatment of the condition and diseases including chronic pain, neuropathic pain, postoperative pain, rheumatoid arthritic pain, neuralgia, neuropathies, algesia, nerve injury, ischaemia, neurodegeneration, stroke, incontinence, inflammatory disorders, urge urinary incontinence (UUI), and/or overactive bladder.

WO 2000/50387 discloses the compounds having a vanilloid agonist activity represented by the general formula: 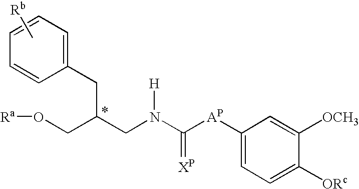
wherein;
X^{P} is an oxygen or sulfur atom;
A^{P} is —NHCH₂— or —CH₂—.
R^{a} is a substituted or unsubstituted C₁₋₄ alkyl group, or R^{a1}CO—;
wherein R^{a1} is an alkyl group having 1 to 18 carbon atoms, an alkenyl group having 2 to 18 carbon atoms, or substituted or unsubstituted aryl group having 6 to 10 carbon atoms;
R^{b} is a hydrogen atom, an alkyl group having 1 to 6 carbon atoms, an alkoxy group having 1 to 6 carbon atoms, a haloalkyl group having 1 to 6 carbon atoms or a halogen atom;
R^{C} is a hydrogen atom, an alkyl group having 1 to 4 carbon atom, an aminoalkyl, a diacid monoester or -alkyl acid; and
the asteric mark * indicates a chiral carbon atom, and their pharmaceutically acceptable salts.

WO 2000/61581 discloses amine derivatives represented by the general formula: 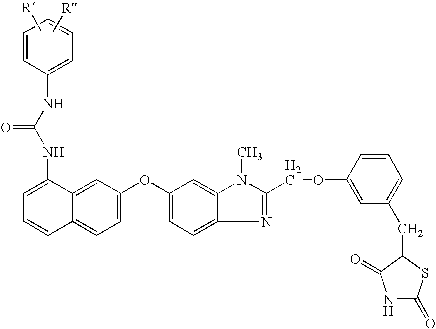
wherein (R′, R″) represent (F, F), (CF₃, H), or (iPr, iPr)
as useful agents for diabetes, hyperlipemia, arteriosclerosis and cancer.

WO 2000/75106 discloses the compounds represented by the general formula: 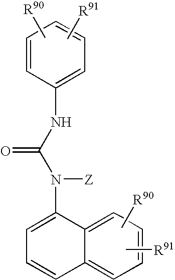
wherein Z represents 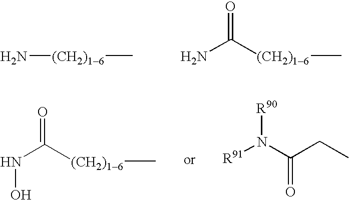
in which R⁹⁰ is hydrogen, C₁₋₁₂ alkyl, C₃₋₈ cycloalkyl, or the like, and R⁹¹ is amino-C₁₋₆ alkyl, aminocarbonyl-C₁₋₆ alkyl, or hydroxyaminocarbonyl C₁₋₆ alkyl; and
R⁹⁰ and R⁹¹ are independently selected from the group consisting of H, C₁₋₆ alkyl, C₁₋₆alkylthio, C₁₋₆ alkoxy, fluoro, chloro, bromo, iodo, and nitro;
   as useful agents for treating MMP-mediated diseases in mammals.

However, none of these reference discloses simple urea derivatives having pharmaceutical activity.

The development of a compound having effective VR1 antagonistic activity and the use of such compound for the prophylaxis and treatment of diseases associated with VR1 activity, in particular for the treatment of urge urinary incontinence and/or overactive bladder have been desired.

### SUMMARY OF THE INVENTION

This invention is to provide urea derivatives of the formula (I), their tautomeric and stereoisomeric form, and salts thereof: 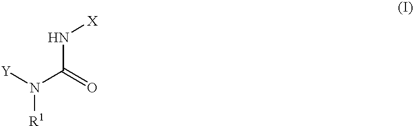
wherein

Y is 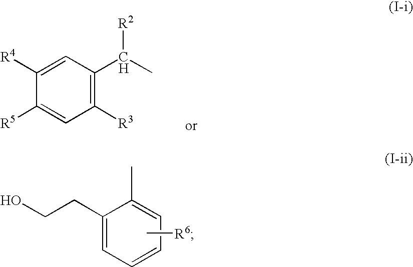
X is C₁₋₆ alkyl substituted by phenyl or naphthyl (wherein said phenyl and naphthyl are optionally substituted by R¹¹, R¹² and R¹³), aryl or heterocyclic ring,
wherein said aryl and heterocyclic ring are optionally substituted by R¹¹, R¹² and R¹³ and are selected from the group consisting of phenyl, naphthyl, pyridyl, carbazolyl, fluorenyl, thienyl, pyrimidyl, benzodioxolyl, indazolyl, and quinolyl,
in which R¹¹, R¹² and R¹³ independently represent hydrogen, halogen, C₁₋₆ alkyl, mono-, di-, or tri-halogen substituted C₁₋₆ alkyl, nitro, cyano, C₁₋₆ alkoxy, hydroxy, piperidino, furyl, thienyl, benzyloxy, anilino, naphthyl, C₁₋₆ alkylcarbamoyl, carbamoyl, carboxyl, amino, C₁₋₆ alkylamino, di(C₁₋₆ alkyl)amino, C₁₋₆ alkoxycarbonyl, benzyl, phenoxy, C₁₋₆ alkyl substituted phenoxy, pyridyl, halogen substituted phenoxy, C₁₋₆ alkylthio, C₁₋₆ alkanoyl, C₁₋₆ alkanoylamino, hydroxy substituted C₁₋₆ alkyl, mono-, di-, or tri-halogen substituted C₁₋₆ alkyloxy, or phenyl optionally substituted by one to three substituents,
in which the substituents are each different or identical and selected from the group consisting of hydrogen, halogen, C₁₋₆ alkyl, C₁₋₆ alkoxy, pyridyl, mono-, di-, or tri-halogen substituted C₁₋₆ alkyl, nitro, cyano, benzyloxy, thienyl, C₁₋₆alkanoyl, C₁₋₆ alkoxycarbonyl, C₁₋₆ alkylthio, di(C₁₋₆ alkyl)amino, and C₁₋₆ alkylamino, mono, di, or tri halogen substituted C₁₋₆ alkyloxy;
R¹ is hydrogen,
R² is hydrogen,
R³ is hydrogen, or
R² and R³ together form —(CH₂)ₘ— (wherein m represents 1, 2, 3 or 4), or
R¹ and R³ together form —(CH₂)ₙ— (wherein n represents 1, 2, or 3);
R⁴ is hydrogen, halogen, C₁₋₆ alkoxy, hydroxy, C₁₋₆ alkoxy substituted benzyloxy, sulfamoyl, C₁₋₆ alkylsulfamoyl, di(C₁₋₆ alkyl)sulfamoyl, di(C₁₋₆ alkyl)aminoC₁₋₆ alkylene sulfamoyl, hydroxy C₁₋₆ alkyl piperazinosulfonyl, C₁₋₆ alkylsulfonylamino, nitro, amino, C₁₋₆ alkanoylamino, C₁₋₆ alkoxyC₁₋₆ alkyleneoxy,
R⁵ is hydrogen, halogen, C₁₋₆ alkoxy, hydroxy, C₁₋₆ alkoxy substituted benzyloxy, sulfamoyl, C₁₋₆ alkylsulfamoyl, di(C₁₋₆ alkyl)sulfamoyl, di(C₁₋₆ alkyl)amino C₁₋₆alkylene sulfamoyl, hydroxy C₁₋₆ alkyl piperazinosulfonyl, C₁₋₆ alkylsulfonylamino, nitro, amino, C₁alkanoylamino, C₁₋₆ alkoxyC₁₋₆ alkyleneoxy, or
R⁴ and R⁵ together form —O—(CH₂)—O—; and
R⁶ is hydrogen, halogen, C₁₋₆ alkyl, mono-, di-, or tri-halogen substituted C₁₋₆ alkyl, nitro, cyano, C₁₋₆ alkoxy, hydroxy, C₁₋₆ alkylcarbamoyl, carbamoyl, carboxyl, amino, C₁₋₆ alkylamino, di(C₁₋₆alkyl)amino, C₁₋₆ alkoxycarbonyl, phenyl, benzyl, phenoxy, halogen substituted phenoxy, C₁₋₆ alkylthio, C₁₋₆ alkanoyl, C₁₋₆ alkanoylamino, hydroxy substituted C₁₋₆ alkyl, mono-, di-, or tri-halogen substituted C₁₋₆ alkoxy.

The urea derivatives of formula (I), their tautomeric and stereoisomeric form, and salts thereof surprisingly show excellent VR1 antagonistic activity. They are, therefore suitable especially for the prophylaxis and treatment of diseases associated with VR1 activity, in particular for the treatment of urge urinary incontinence and/or overactive bladder.

Alkyl per se and “alk” and “alkyl” in alkoxy, alkanoyl, alkylthio, alkylamino, alkylaminocarbonyl, alkylaminosulphonyl, alkylsulphonylamino, alkoxycarbonyl, alkoxycarbonylamino, alkylcarbamoyl and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, iso-propoxy, tert-butoxy, n-pentoxy and n-hexoxy.

Alkanoyl illustratively and preferably represents acetyl and propanoyl.

Alkylamino represents an alkylamino radical having one or two (independently selected) alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkylaminocarbonyl or alkylcarbamoyl represents an alkylaminocarbonyl radical, having one or two (independently selected) alkyl substituents, illustratively and preferably representing methylaminocarbonyl, ethylaminocarbonyl, n-propylamino-carbonyl, isopropylamino-carbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methylaminocarbonyl.

Alkoxycarbonyl illustratively and preferably represents methoxycarbonyl, ethoxycarbonyl, n-propoxycarbonyl, isopropoxycarbonyl, tert-butoxycarbonyl, n-pentoxycarbonyl and n-hexoxycarbonyl. Alkoxycarbonylamino illustratively and preferably represents methoxycarbonylamino, ethoxycarbonylamino, n-propoxycarbonylamino, isopropoxycarbonylamino, tert-butoxycarbonylamino, n-pentoxycarbonylamino and n-hexoxycarbonylamino.

Alkanoylamino illustratively and preferably represents acetylamino and ethylcarbonylamino.

Halogen represents fluorine, chlorine, bromine and iodine.

Aryl per se and in arylamino and in arylcarbonyl represents a mono- to tricyclic aromatic carbocyclic radical having generally 6 to 14 carbon atoms, and more preferably from 6-10 carbon atoms, optionally substituted with one or more substituents. Examples of aryl radicals include, but are not limited to phenyl, naphthyl, indenyl, azulenyl, fluorenyl, anthracenyl, biphenyl, fluorenonyl and the like.

Heterocyclic ring refers to a 3- to 15-membered ring radical which consists of carbon atoms and from one to five heteroatoms selected from the group consisting of nitrogen, oxygen and sulfur. The heterocyclic ring radical may be a monocyclic, bicyclic or tricyclic ring system, which may include fused or bridged ring systems and may be partially or fully saturated or aromatic. Examples of such rings include, but are not limited to thienyl, benzothienyl, furanyl, benzofuranyl, pyrazinyl, pyrazolyl, pyridazinyl, pyridyl, pyrimidinyl, pyrrolyl, isothiazolyl, thiazolyl, oxazolyl, isoxazolyl, triazolyl, tetrazolyl, imidazolyl, thiadiazolyl, benzothiadiazolyl, oxadiazolyl, benzothiazolyl, indolyl, carbazolyl, quinolinyl, isoquinolinyl, benzodioxolyl, indazolyl, indazolinolyl and the like.

This invention is also to provide a method for treating or preventing a disorder or disease associated with VR1 activity in a human or animal subject, comprising administering to said subject a therapeutically effective amount of the urea derivative shown in the formula (I), its tautomeric or stereoisomeric form, or a physiologically acceptable salt thereof.

Further this invention is to provide a use of the urea derivative shown in the formula (I), its tautomeric or stereoisomeric form, or a physiologically acceptable salt thereof in the preparation of a medicament. Preferably, said medicament is suitable for treating or preventing a disorder or disease associated with VR1 activity.

The compounds of the present invention surprisingly show excellent VR1 activity. They are, therefore, suitable for the production of medicament or medical composition, which may be useful t, treat VR1 related diseases.

More specifically, since the urea derivatives of the present invention inhibit VR1, they are useful for treatment and prophylaxis of diseases as follows:
urinary incontinence, overactive bladder, chronic pain, neuropathic pain, post-operative pain, rheumatoid arthritic pain, neuralgia, neuropathies, algesia, nerve injury, ischaemia, neurodegeneration, stroke, incontinence and inflammatory disorders.

In one embodiment, the compounds of formula (I) are those wherein:

Y is I-i;
X is phenyl optionally substituted by R¹¹, R¹² and R¹³, phenyl C₁₋₆ alkyl (wherein said phenyl is optionally substituted by R¹¹, R¹² and R¹³), or naphthyl optionally substituted by R¹¹, R¹² and R¹³,
   in which R¹¹, R¹² and R¹³ independently represent hydrogen, halogen, C₁₋₆ alkyl, mono-, di-, or tri-halogen substituted C₁₋₆ alkyl, nitro, C₁₋₆ alkoxy, C₁₋₆ alkoxycarbonyl, phenoxy, C₁₋₆ alkylthio, or C₁₋₆ alkanoyl.

In another embodiment; the compounds of formula (I) are those wherein:

Y is I-i;
R¹ is hydrogen;
R² is hydrogen; and
R³ is hydrogen.

In another embodiment, the compounds of formula (I) are those wherein:

Y is I-i;
X is phenyl optionally substituted by R¹¹, R¹² and R¹³, phenyl C₁₋₆ alkyl (wherein said phenyl is optionally substituted by R¹¹, R¹² and R³), or naphthyl optionally substituted by R¹¹, R¹² and R¹³,
   in which R¹¹, R¹² and R¹³ independently represent hydrogen, halogen, C₁₋₆ alkyl, mono-, di-, or tri-halogen substituted C₁₋₆ alkyl, nitro, C₁₋₆ alkoxy, C₁₋₆ alkoxycarbonyl, phenoxy, C₁₋₆ alkylthio, or C₁₋₆ alkanoyl.
R¹ is hydrogen; and
R² and R³ together form —(CH₂)ₘ— (wherein m represents 1, 2, 3 or 4).

In another embodiment, the compounds-of formula (I) are those wherein:

Y is I-i;
R¹ and R³ together form —(CH₂)ₙ— (wherein n represents 1, 2, or 3) and
R² is hydrogen.

alternatively, the urea derivative of formula (f) can be those wherein:

Y is I-ii;
R⁶ is hydrogen, halogen, C₁₋₆ alkyl, mono-, di-, or tri-halogen substituted C₁₋₆ alkyl, phenyl, or C₁₋₆ alkoxy.

In another embodiment, the compounds of formula (I) are those wherein:
Y is 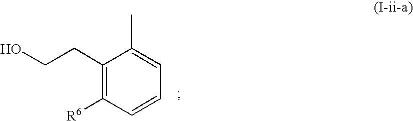
X is C₁₋₆ alkyl substituted by phenyl or naphthyl (wherein said phenyl and naphthyl are optionally substituted by R¹¹, R¹² and R¹³), aryl or Heterocyclic ring,
   wherein said aryl and Heterocyclic ring are optionally substituted by R¹¹, R¹² and R¹³ and are selected from the group consisting of phenyl, naphthyl, pyridyl, carbazolyl, fluorenyl, thienyl, benzodioxolyl, indazolyl, and quinolyl,
R⁶ is hydrogen, halogen, C₁₋₆ alkyl, mono-, di-, or tri-halogen substituted C₁₋₆ alkyl, phenyl, or C₁₋₆ alkoxy.

The preferable compounds of the present invention are as follows:
N-(4-hydroxy-3-methoxybenzyl)-N′-(4-isopropylphenyl)urea;
N-(4-hydroxy-3-methoxybenzyl)-N′-(1-naphthyl)urea;
N-(3,4-dichlorophenyl)-N′-(4-hydroxy-3-methoxybenzyl)urea;
N-(3-chloro-4-methylphenyl)-N′-(4-hydroxy-3 methoxybenzyl)urea;
N-(4-hydroxy-3-methoxybenzyl)-N′-(4-phenoxyphenyl)urea;
N-[2-chloro-5-(trifluoromethyl)phenyl]-N′-(4-hydroxy-3-methoxybenzyl)urea;
N-(3-chlorophenyl)-N′-(4-hydroxy-3-methoxybenzyl)urea;
N-(4-chlorophenyl)-N′-(4-hydroxy-3-methoxybenzyl)urea;
N-[4-chloro-3-(trifluoromethyl)phenyl]-N′-(4-hydroxy-3-methoxybenzyl)urea;
N-(4′-chloro-1,1′-biphenyl-3-yl)-N′-(4-hydroxy-3-methoxybenzyl)urea;
N-[2-(2-hydroxyethyl)phenyl]-N′-[4′-(methylsulfanyl)-1,1′-biphenyl-3-yl]urea;
N-[2-(2-hydroxyethyl)phenyl]-N′-(4′-nitro-1,1′-biphenyl-3-yl)urea;
N-(4′-acetyl-1,1′-biphenyl-3-yl)-N′-[2-(2-hydroxyethyl)phenyl]urea;
Ethyl3′-[({[2-(2-hydroxyethyl)phenyl]amino}carbonyl)amino]-1,1′-biphenyl-4-carboxylate;
N-[2-(2-hydroxyethyl)phenyl]-N′-[2′-(trifluoromethyl)-1,1′-biphenyl-3-yl]urea;
N-(2′-chloro-1,1′-biphenyl-3-yl)-N′-[2-(2-hydroxyethyl)phenyl]urea;
N-[2-(2-hydroxyethyl)phenyl]-N′-[3-(1-naphthyl)phenyl]urea;
N-[2-(2-hydroxyethyl)phenyl]-N′-[4′-(trifluoromethyl)-1,1′-biphenyl-3-yl]urea;
N-(4′,6-dichloro-1,1′-biphenyl-3-yl)-N′-[2-(2-hydroxyethyl)phenyl]urea;
N-(2′,5′-dichloro-1,1′-biphenyl-3-yl)-N′-[2-(2-hydroxyethyl)phenyl]urea;
N-(2′,4′-dichloro-1,1′-biphenyl-3-yl)-N′-[2-(2-hydroxyethyl)phenyl]urea;
N-(3′,4′-difluoro-1,1′-biphenyl-3-yl)-N′-[2-(2-hydroxyethyl)phenyl]urea;
N-(4′-fluoro-1,1′-biphenyl-3-yl)-N′-[2-(2-hydroxyethyl)phenyl]urea;
N-[2-(2-hydroxyethyl)phenyl]-N′-(3′-nitro-1,1′-biphenyl-3-yl)urea;
N-[4′-(benzyloxy)-3′-fluoro-1,1′-biphenyl-3-yl]-N′-[2-(2-hydroxyethyl)phenyl]urea;
N-(4′-chloro-1,1′-biphenyl-3-yl)-N′-[2-(2-hydroxyethyl)phenyl]urea;
N-(2′,5′-dimethyl-1,1′-biphenyl-3-yl)-N′-[2-(2-hydroxyethyl)phenyl]urea;
N-[2-(2-hydroxyethyl)phenyl]-N′-[4′-(trifluoromethoxy)-1,1′-biphenyl-3-yl]urea;
N-(4′-chloro-1,1′-biphenyl-3-yl)-N′-[2-(2-hydroxyethyl)-3-methoxyphenyl]urea;
N-(3′-fluoro-1,1′-biphenyl-3-yl)-N′-[2-(2-hydroxyethyl)phenyl]urea;
N-(3′-chloro-1,1′-biphenyl-3-yl)-N′-[2-(2-hydroxyethyl)phenyl]urea;
N-(2′,5′-difluoro-1,1′-biphenyl-3-yl)-N′-[2-(2-hydroxyethyl)phenyl]urea; and
N-(3′-chloro-4′-fluoro-1,1′-biphenyl-3-yl)-N′-[2-(2-hydroxyethyl)phenyl]urea.

Preferably, the medicaments of the present invention further comprise one or more pharmaceutically acceptable carrier and/or excipients.

### EMBODIMENT OF THE INVENTION

The compound of the formula (I) of the present invention can be, but not limited to be, prepared by either of the methods [A], [B] and [C] below. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

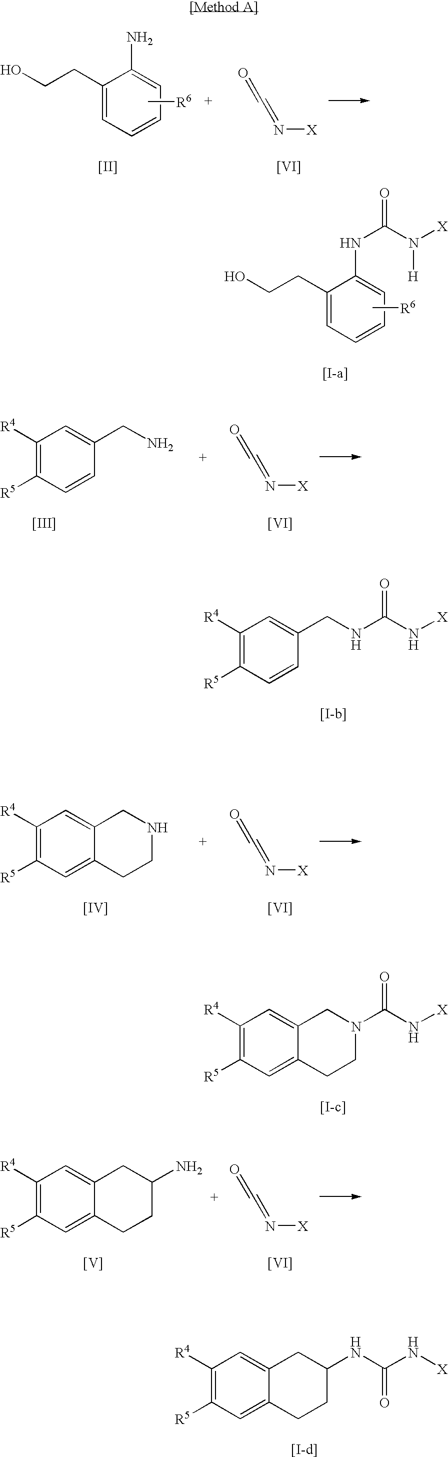

The compound [I-a] wherein X and R⁶ are the same as defined above, can be prepared by the reaction of a substituted 2-(2-aminophenyl)ethanol[II] (wherein R⁶ is the same as defined above) and isocyanate of the formula [VI] (wherein X is the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); urea such as 1,3-dimethyl-2-imidazolidinone (DMI); sulfoxides such as dimethylsulfoxide (DMSO); and others.

The reaction temperature can be optionally set depending on the compounds to be reacted. The reaction temperature is usually, but not limited to, about 30° C. to 100° C. The reaction may be conducted for, usually, 30 minutes to 48 hours and preferably 1 to 24 hours.

The compound [I-b], [I-c] and [I-d] wherein X, R⁴ and R⁵ are the same as defined above, can be prepared using substituted benzylamines [III], substituted tetrahydro-isoquinolines [IV] and substituted tetrahydro-naphthalenylamine [V] as starting material, respectively, by the same method as for the compound [I-a].

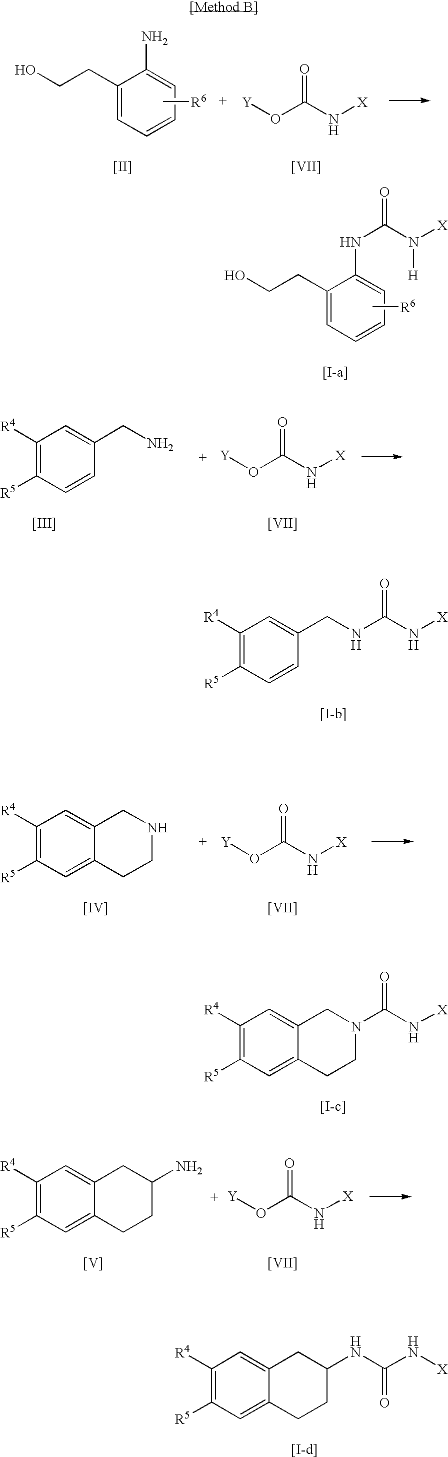

Alternatively, the compound [I-a] (wherein X and R⁶ are the same as defined above) can be prepared by reacting a substituted 2-(2-aminophenyl)ethanol[II] and carbamate of the formula [VII] (wherein X is the same as defined above and Y represents phenyl).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (MP); urea such as 1,3-dimethyl-2-imidazolidinone (DMI); sulfoxides such as dimethylsulfoxide (DMSO); and others.

The reaction temperature can be optionally set depending on the compounds to be reacted. The reaction temperature is usually, but not limited to, about 20° C. to 100° C. The reaction may be conducted for, usually, 30 minutes to 40 hours and preferably 1 to 24 hours.

The compound [I-b], [I-c] and [I-d] wherein X, R⁴ and R⁵ are the same as defined above, can be prepared using substituted benzylamines [III], substituted tetrahydro-isoquinolines [IV] and substituted tetrahydro-naphthalenylamine [V] as starting material, respectively, by the same method as for the compound [I-a].

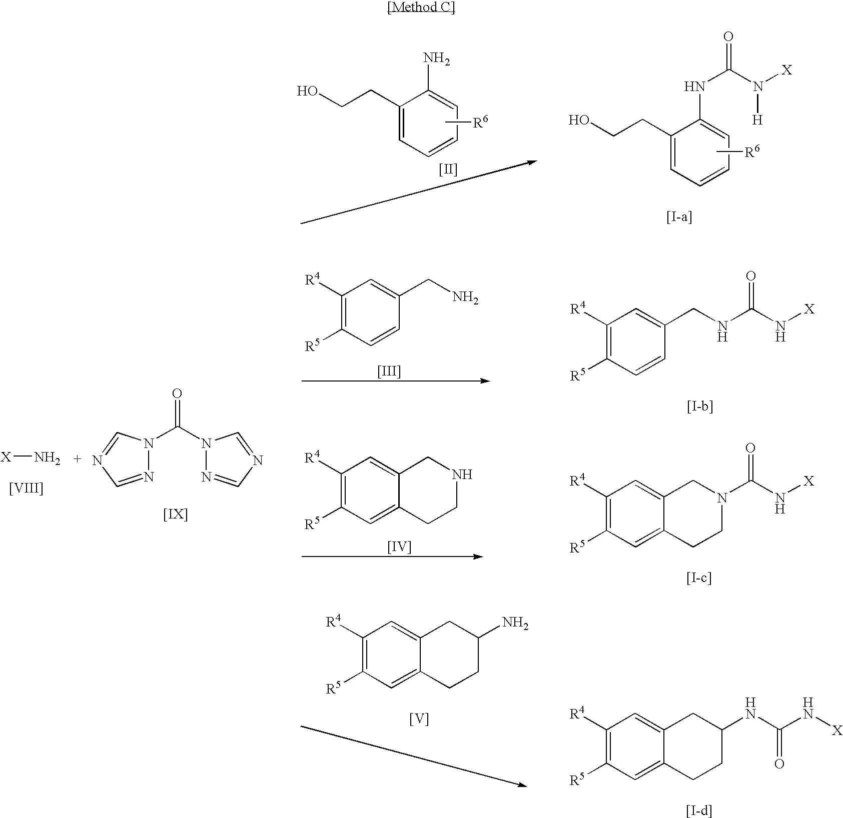

The compound [I-a] can be prepared by reacting amine of the formula [VIII](wherein X is the same as defined above) and 1,1′-carbonyldi(1,2,4-triazole) (CDT)[IX], and then adding substituted 2-(2-aminophenyl)ethanol[II] to the reaction mixture. The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitrites such as acetonitrile; amides such as N,N-dimethylformamide (DMF), N,N-dimethylacetamide (DMAC) and N-methylpyrrolidone (NMP); urea such as 1,3-dimethyl-2-imidazolidinone (DMI); sulfoxides such as dimethylsulfoxide (DMSO); and others.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The compound [I-b], [I-c] and [I-d] wherein X, R⁴ and R⁵ are the same as defined above, can be prepared using substituted benzylamines [III], substituted tetrahydro-isoquinolines [IV] and substituted tetrahydro-naphthalenylamine. [V] as starting material, respectively, by the same method as for the compound [I-a].

The substituted 2-(2-aminophenyl)ethanols [II], substituted benzylamines [III], substituted tetrahydroisoquinolines [IV], substituted tetrahydro-naphthalenylamine [V], Isocyanates [VI], carbamates [VII], amine [VIII] and CDT [IX] are commercially available or can be prepared by the use of known techniques or by method described in the examples.

When the compound shown by the formula (I) or a salt thereof has tautomeric isomers and/or stereoisomers (e.g., geometrical isomers and conformational isomers), each of their separated isomer and mixtures are also included in the scope of the present invention.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, respectively.

Acids to form acid addition salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic, acid, p-bromophenylsulfonic acid, carbonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include, sodium hydroxide, potassium hydroxide, potassium carbonate, sodium carbonate, sodium bicarbonate, potassium bicarbonate, calcium hydroxide, calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients therefore. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and the like; together with, optionally, disintegrating agents, such as, without limitation, maize, starch, methyl cellulose, agar, bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without, limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient, may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

The formulation may be in unit dosage form, which is a physically discrete unit containing a unit dose, suitable for administration in human or other mammals. A unit dosage form can be a capsule or tablets, or a number of capsules or tablets. An “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.0 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described as a form of examples, but they should by no means be construed as defining the metes and bounds of the present invention.

In the examples below, all quantitative data, if not stated otherwise, relate to percentages by weight.

Mass spectra were obtained using electrospray (ES) ionization techniques (micromass Platform LC). Melting points are uncorrected. Liquid Chromatography-Mass spectroscopy (LC-MS) data were recorded on a Micromass Platform LC with Shimadzu Phenomenex ODS column (4.6 mm×30 mm) flushing a mixture of acetonitrile-water (9:1 to 1:9) at 1 ml/min of the flow rate. TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Tokyo kasei kogyo co. Ltd., Arch corporation.

The effect of the present compounds were examined by the following assays and pharmacological tests.

[Measurement of Capsaicin-Induced Ca²⁺ Influx in the Human VR1-Transfected CHO Cell Line] (Assay 1)

(1) Establishment of the Human VR1-CHOluc9aeq Cell Line

Human vanilloid receptor (hVR1) cDNA was cloned from libraries of axotomized dorsal root ganglia (WO2000/29577). The cloned hVR1 cDNA was constructed with pcDNA3 vector and transfected into a CHOluc9aeq cell line. The cell line contains aequorin and CRE-luciferase reporter genes as read-out signals. The transfectants were cloned by limiting dilution in selection medium (DMEM/F12 medium (Gibco BRL) supplemented with 10% FCS, 1.4 mM Sodium pyruvate, 20 mM HEPES, 0.15% Sodium bicarbonate, 100 U/ml penicillin, 100 μg/ml streptomycin, 2 mM glutamine, non-essential amino acids and 2 mg/ml G418). Ca²⁺ influx was examined in the capsaicin-stimulated clones. A high responder clone was selected and used for further experiments in the project. The human VR1-CHOluc9aeq cells were maintained in the selection medium and passaged every 3-4 days at 1-2.5×10⁵ cells/flask (75 mm²).

(2) Measurement of Ca²⁺ Influx Using FDSS-3000

Human VR1-CHOluc9aeq cells were suspended in a culture medium which is the same as the selection medium except for G418 and seeded at a density of 1,000 cells per well into 384-well plates (black walled clear-base/Nalge Nunc International). Following the culture for 48 hrs the medium was changed to 2 μM Fluo-3 AM (Molecular Probes), and 0.02% Puronic F-127 in assay buffer (Hank's balanced salt solution (HBSS), 17 mM HEPES (pH7.4), 1 mM Probenecid, 0.1% BSA) and the cells were incubated for 60 min at 25° C. After washing twice with assay buffer the cells were incubated with a test compound or vehicle for 20 min at 25° C. Mobilization of cytoplasmic Ca²⁺ was measured by FDSS-3000 (λₑₓ=488 nm, λₑₘ=540 nm/Hamamatsu Photonics) for 60 sec after the stimulation with 10 nM capsaicin. Integral R was calculated and compared with controls.

[Measurement of the Capsaicin-Induced Ca²⁺ Influx in Primary Cultured Rat Dorsal Root Ganglia Neurons] (Assay 2)

(1) Preparation of Rat Dorsal Root Ganglia Neurons

New born Wister rats (5-11 days) were sacrificed and dorsal root ganglia (DRG) was removed. DRG was incubated with 0.1% trypsin (Gibco BRL) in PBS(−) (Gibco BRL) for 30 min at 37° C., then a half volume of fetal calf serum (FCS) was added and the cells were spun down. The DRG neuron cells were resuspended in Han F12/5% FCS/5% horse serum (Gibco BRL) and dispersed by repeated pipetting and passing through 70 μm mesh (Falcon). The culture plate was incubated for 3 hrs at 37° C. to remove contaminating Schwann cells. Non-adherent cells were recovered and further cultured in laminin-coated 384 well plates (Nunc) at 1×10⁴ cells/50 μl/well for 2 days in the presence of 50 ng/ml recombinant rat NGF (Sigma) and 50 μM 5-fluoro-deoxyuridine (Sigma).

(2) Ca²⁺ Mobilization Assay

DRG neuron cells were washed twice with HBSS supplemented with 17 mM HEPES (PH 7.4) and 0.1% BSA. After incubating with 2 μM fluo-3AM (Molecular Probe)., 0.02% PF127 (Gibco BRL) and 1 mM probenecid (Sigma), for 40 min at 37° C., cells were washed 3 times. The cells were incubated with VR1 antagonists or vehicle (dimethylsulphoxide) and then with 1 μM capsaicin in FDSS-6000 (λₑₓ=480 nm, λₑₘ=520 nm/Hamamatsu Photonics). The fluorescence changes at 480 nm were monitored for 2.5 min. Integral R was calculated and compared with controls.

[Organ Bath Assay to Measure the Capsaicin-Induced Bladder Contraction] (Assay 3)

Male Wistar rats (10 week old) were anesthetized with ether and sacrificed by dislocating the necks. The whole urinary bladder was excised and placed in oxygenated Modified Krebs-Henseleit solution (pH 7.4) of the following composition (12 mM NaCl, 5.9 mM KCl, 1.2 mM MgCl₂, 1.2 mM NaH₂PO₄, 2 mM CaCl₂, 2.5 mM NaHCO₃, 12 mM glucose). Contractile responses of the urinary bladder were studied as described previously [Maggi C A et al: Br. J. Pharmacol. 108: 801-805, 1993]. Isometric tension was recorded under a load of 1 g using longitudinal strips of rat detrusor muscle. Bladder strips were equilibrated for 60 min before each stimulation. Contractile response to 80 mM KCl was determined at 15 min intervals until reproducible responses were obtained. The response to KCl was used as an internal standard to evaluate the maximal response to capsaicin. The effects of the compounds were investigated by incubating the strips with compounds for 30 min prior to the stimulation with 1 μM capsaicin (vehicle: 80% saline, 10% EtOH, and 10% Tween 80). One of the preparations made from the same animal was served as a control while the others were used for evaluating compounds. Ratio of each capsaicin-induced contraction to the internal standard (i.e. KCl-induced contraction) was calculated and the effects of the test compounds on the capsaicin-induced contraction were evaluated.

[Measurement of Ca⁺ Influx in the Human P2X1-Transfected CHO Cell Line]

(1) Preparation of the Human P2X1-Transfected CHOluc9aeq Cell Line

Human P2X1-transfected CHOluc9aeq cell line was established and maintained in Dulbecco's modified Eagle's medium (DMEM/F12) supplemented with 7.5% FCS, 20 mM HEPES-KOH (pH 7.4), 1.4 mM sodium pyruvate, 100 U/ml penicillin, 100 μg/ml streptomycin, 2 mM glutamine (Gibco BRL) and 0.5 Units/ml apyrase (grade I, Sigma). The suspended cells were seeded in each well of 384-well optical bottom black plates (Nalge Nunc International) at 3×10³/50 μl/well. The cells were cultured for following 48 hrs to adhere to the plates.

(2) Measurement of the Intracellular Ca²⁺ Levels

P2X1 receptor agonist-mediated increases in cytosolic Ca²⁺ levels were measured using a fluorescent Ca²⁺ chelating dye, Fluo-3 AM (Molecular Probes). The plate-attached cells were washed twice with washing buffer (HBSS, 17 mM HEPES-KOH (pH 7.4), 0.1% BSA and 0.5 units/ml apyrase), and incubated in 40 μl of loading buffer (1 μM Fluo-3 AM, 1 mM probenecid, 1 μM cyclosporin A, 0.01% pluronic (Molecular Probes) in washing buffer) for 1 hour in a dark place. The plates were washed twice with 40 μl washing buffer and 35 μl of washing buffer were added in each well with 5 μl of test compounds or 2,3′-o-(2,4,6-trinitrophenyl)adenosine 5′-triphpsphate (Molecular Probes) as a reference. After further incubation for 10 minutes in dark 200 nM α,β-methylene ATP agonist was added to initiate the Ca²⁺ mobilization. Fluorescence intensity was measured by FDSS-6000 (λₑₓ=410 nm, λₑₘ=510 nm/Hamamatsu Photonics) at 250 msec intervals. Integral ratios were calculated from the data and compared with that of a control.

[Measurement of Capsaicin-Induced Bladder Contraction in Anesthetized Rats] (Assay 4)

(15 Animals

Female Sprague-Dawley rats (200250 g/Charles River Japan) were used.

(2) Catheter Implantation

Rats were anesthetized by intraperitoneal administration of urethane (Sigma) at 1.2 g/kg. The abdomen was opened through a midline incision, and a polyethylene catheter (BECTON DICKINSON, PE50) was implanted into the bladder through the dome. In parallel, the inguinal region was incised, and a polyethylene catheter (Hibiki, size 5) filled with 2 IU/ml of heparin (Novo Heparin, Aventis Pharma) in saline (Otsuka) was inserted into a common iliac artery.

(3) Cystometric Investigation

The bladder catheter was connected via T-tube to a pressure transducer (Viggo-Spectramed Pte Ltd, DT-XXAD) and a microinjection pump (TERUMO). Saline was infused at room temperature into the bladder at a rate of 2.4 ml/hr. Intravesical pressure was recorded continuously on a chart pen recorder (Yokogawa). At least three reproducible micturition cycles, corresponding to a 20-minute period, were recorded before a test compound administration and used as baseline values.

(4) Administration of Test Compounds and Stimulation of Bladder with Capsaicin

The saline infusion was stopped before administrating compounds. A testing compound dissolved in the mixture of ethanol, Tween 80 (ICN Biomedicals Inc.) and saline (1:1:8, v/v/v) was administered intraarterially at 10 mg/kg. 2 min after the administration of the compound 10 μg of capsaicin (Nacalai Tesque) dissolved in ethanol was administered intraarterially.

(5) Analysis of Cystometry Parameters

Relative increases in the capsaicin-induced intravesical pressure were analyzed from the cystometry data. The capsaicin-induced bladder pressures were compared with the maximum bladder pressure during micturition without the capsaicin stimulation. The testing compounds-mediated inhibition of the increased bladder pressures was evaluated using Student's t-test. A probability level less than 5% was accepted as significant difference.

Results of IC₅₀ of capsaicin-induced Ca²⁺ influx in the human VR1-transfected CHO cell line are shown in Examples and tables of the Examples below. The data corresponds to the compounds as yielded by solid phase synthesis and thus to levels of purity of about 40 to 90%. For practical reasons, the compounds are grouped in four classes of activity as follows:
IC₅₀=A 0.1 μM<B 0.5 μM<C 1 μM<D

The compounds of the present invention also show excellent selectivity, and strong activity in other assays (2)-(4) described above.

Preparing Method of Starting Compounds:

[Starting Compound A]

### 7-methoxy-1,2,3,4-tetrahydro-6-isoquinolinol

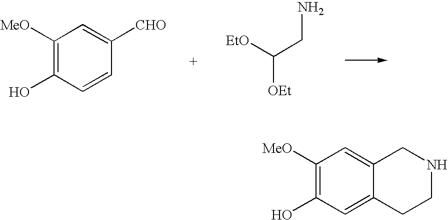

An ethanol (15 ml) solution of aminoacetaldehyde-diethyl acetal (2.66 g, 20.0 mmol) and vanillin (3.04 g, 20.0 mmol) was added to a suspension of platinum (prepared by reduction of 0.2 g of platinum oxide) in ethanol (20 ml). The mixture was stirred under a hydrogen atmosphere at room temperature for 4 hrs. The catalyst was removed and the solvent was evaporated under reduced pressure. The residue was dissolved in 6N HCl (150 ml) and Pd/C (2.0 g, 10%) was added. The reaction mixture was stirred under a hydrogen atmosphere at room temperature for 16 hrs. The catalyst was removed by filtration and the filtrate was concentrated under reduced pressure. The residue was collected and washed with ethanol to give 7-methoxy-1,2,3,4-tetrahydro-6-isoquinolinol (0/75 g, 25%).

[Starting Compound B]

### 6-methoxy-1,2,3,4-tetrahydro-7-isoquinolinol

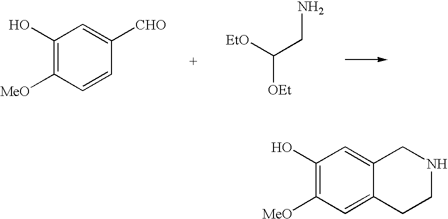

Starting material B was prepared by the same method as for starting material A, using isovanillin instead of vanillin. 6-methoxy-1,2,3,4-tetrahydro-7-isoquinolinol (0.03 g, 35%).

[Starting Compound C]

### 7-nitro-1,2,3,4-tetrahydro-1-naphthalenamine

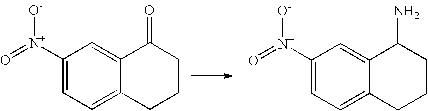

A mixture of 7-nitro-1-tetralone (1.91 g, 10.0 mmol), titanium (IV)tetraisopropoxide (5.9 ml, 20.0 mmol), ammonium chloride (1.07 g, 20.0 mmol) and triethylamine (2.8 ml, 20.0 mmol) in ethanol (20 ml) was stirred for 16 hrs at room temperature. Sodium tetrahydroborate (0.57 g, 15.0 mmol) was added and the reaction mixture was stirred for another 7 hrs at room temperature. 2M aqueous ammonia (30 ml) was added and after filtration of the inorganic precipitate, extraction was carried out with diethylether. The organic layer was then extracted with 2M HCl. The HCl solution was washed with diethylether and then treated with 2M NaOH. Extraction with diethylether was carried out. The organic layer was washed with brine, dried over Na₂SO₄ and then concentrated to give 7-nitro-1,2,3,4-tetrahydro-1-naphthalenamine (0.25 g, 20%).

[Starting Compound D]

### 4-(aminomethyl)-1-methoxy-2-[(4-methoxybenzyl)oxy]benzene

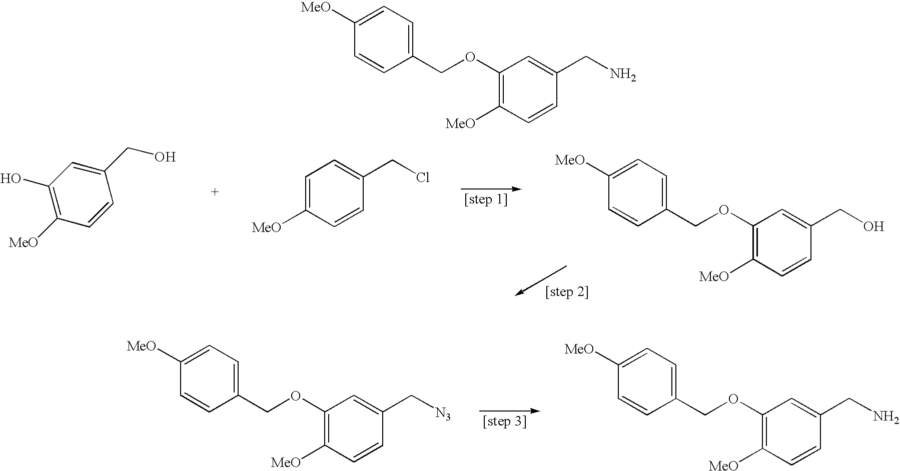 [0170] Step 1: To a suspension of 3-hydroxy-4-methoxybenzyl alcohol (2.00 g, 13.0 mmol) and K₂CO₃ (20.13 g, 13.6 mmol) in acetone (80 ml) was added methoxybenzylchloride (2.13 g, 13.6 mmol). The reaction mixture was stirred at 60° C. for 16 hrs. The mixture was concentrated under reduced pressure and the residue was dissolved in AcOEt/water. Extraction was carried out with AcOEt and the organic layer was washed with brine, dried over Na₂SO₄ and then concentrated under reduced pressure to give {4-methoxy-3-[(4-methoxybenzyl)oxy]phenyl}methanol (quantitative yield).
Step 2: To a mixture of {4-methoxy-3-[(4-methoxybenzyl)oxy]phenyl}methanol (1.00 g, 3.7 mmol) and 1,8-diazabicyclo[5.4.0]undec-7-ene (0.61 g, 4.0 mmol) in toluene (18 ml) was added diphenylphosphinyl azide (1.10 g, 4.0 mmol) at 0° C. The mixture was stirred at room temperature for 4 hrs. Water was added and extraction was carried out with AcOEt. The organic layer was washed with brine, dried over Na₂SO₄ and concentrated under reduced pressure. The residue was passed through a silica gel plug (hexane:AcOEt=1:1) and the filtrate was concentrated under reduced pressure to give 4-(azidomethyl)-1-methoxy-2-[(4-methoxybenzyl)oxy]benzene (1.00 g, 92%) which was used for the next step without any further purification.
Step 3: To a solution of 4-(azidomethyl)-1-methoxy-2-[(4-methoxybenzyl)oxy]benzene (1.00 g, 3.3 mmol) in THF (33 ml) was added triphenylphosphine (2.63 g, 10.0 mmol) and water (0.25 ml) at room temperature. The reaction mixture was stirred at room temperature for 16 hrs and then concentrated under reduced pressure. The residue 4-(aminomethyl)-1-methoxy-2-[(4-methoxybenzyl)oxy]benzene was used in the reaction with isocyanates following method A without any further purification.
   [Starting Compound E]

### (3-{[tert-butyl(dimethyl)silyl]oxy}-4-methoxyphenyl)methanamine

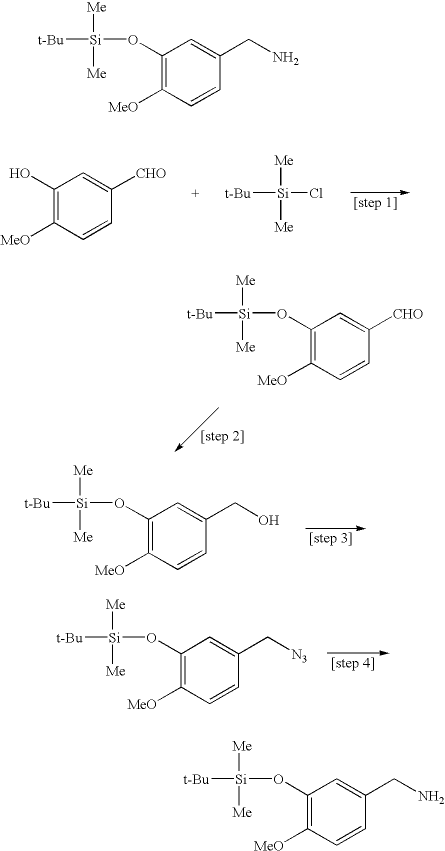 [0174] Step 1: To a solution of 3-hydroxy-4-methoxybenzaldehyde (3.00 g, 19.7 mmol) and imidazole (1.61 g, 23.7 mmol) in DMF (40 ml) was added t-butyldimethylsilylchloride (3.12 g, 20.7 mmol) at 0° C. The reaction mixture was stirred at room temperature for 4 hrs and then diluted with diethylether. The organic layer was washed with brine, dried over Na₂SO₄ and then concentrated under reduced pressure. The residue product was used in the next step without any further purification.
Step 2: To a solution of 3-{[tert-butyl(dimethyl)silyl]oxy}-4-methoxybenzaldehyde (5.25 g, 19.7 mmol) was added NaBH₄ (0.75 g, 19.7 mmol) and the reaction mixture was stirred at room temperature for 16 hrs. Saturated NH₄Cl solution was added and the solvent was removed under reduced pressure. The residue was extracted with AcOEt and the organic layer was washed with brine and dried over Na₂SO₄ and then concentrated under reduced pressure. The residue was purified by silica gel column chromatography (Hexane:AcOEt=9:1-3:1) to give (3-{[tertbutyl(dimethyl)silyl]oxy}-4-methoxyphenyl)methanol (4.51 g, 85%).
Step 3: To a mixture of (3-{[tert-butyl(dimethyl)silyl]oxy}-4-methoxyphenyl)methanol (1.00 g, 3.7 mmol) and 1,8-diazabicyclo[5.4.0]undec-7-ene (0.60 g, 3.9 mmol) in toluene (18 ml) was added diphenylphosphinyl azide (1.08 g, 3.9 mmol) at 0° C. The mixture was stirred at room temperature for 4 hrs. Water was added and extraction was carried out with AcOEt. The organic layer was washed with brine, dried over Na₂SO₄ and concentrated under reduced pressure. The residue was passed through a silica gel plug (hexane:AcOEt=1) and the filtrate was concentrated under reduced pressure to give [5-(azidomethyl)-2-methoxyphenoxy](tert-butyl)dimethylsilane (1.09 g, quantitative) which was used for the next step without any further purification.
Step 4: To a solution of [5-(azidomethyl)-2-methoxyphenoxy](tert-butyl)dimethylsilane (1.09 g, 3.7 mmol) in AcOEt (20 ml) was added 10% Pd/C (0.10 g) and the reaction mixture was stirred at room temperature for one day under a hydrogen atmosphere. The catalyst was removed by filtration and the filtrate was concentrated under reduced pressure. The residue was washed with diisopropyl ether and hexane to give (3-{[tert-butyl(dimethyl)silyl]oxy}-4-methoxyphenyl)methanamine which was used in the next step without any further purification.
   [Starting Compound F]

### [3-(methoxymethoxy)phenyl]methanamine

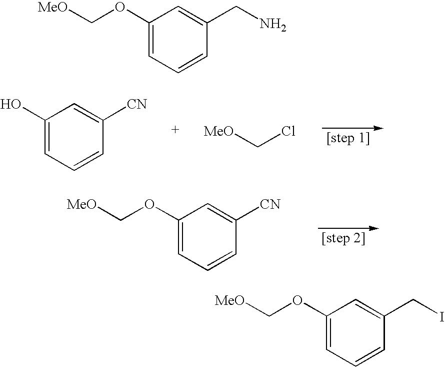 [0179] Step 1: To a solution of 3-hydroxybenzonitrile (5.00 g, 42.0 mmol) and N,N-diisopropylethylamine (8.14 g, 63.0 mmol) in CH₂Cl₂ (100 ml) was added chlorodimethyl ether (4.06 g, 50.4 mmol) at 0° C. The reaction temperature was allowed to rise to room temperature over 30 minutes. The mixture was then stirred at room temperature for 3 hrs. The mixture was then washed with water, dried over Na₂SO₄ and then concentrated under reduced pressure. 3-(methoxymethoxy)benzonitrile (4.24 g, 62%) was obtained as clear oil.
Step 2: To a cooled (0° C.) suspension of lithiumaluminiumhydride (0.84 g, 22.1 mmol) in THF (50 ml) was added dropwise a solution of 3-(methoxymethoxy) benzonitrile (3.00 g, 18.4 mmol) in THF (10 ml). The reaction mixture was stirred at 0° C. for 1 hr and then at room temperature for 3 hrs. A 5 N NaOH solution was added dropwise at 0° C. and the resulting precipitate was filtered off. The filtrate was concentrated under reduced pressure and the residue was dissolved in AcOEt. This was washed with water, dried over Na₂SO₄, and then concentrated under reduced pressure to give [3-(methoxymethoxy)phenyl]methanamine (1.78 g, 58%).
   [Starting Compound G]

### 8-amino-5,6,7,8-tetrahydro-2-naphthalenol

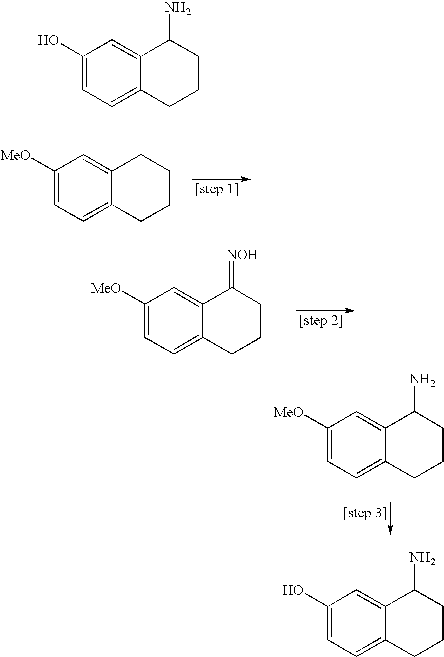 [0182] Step 1: A mixture of 7-methoxy-1-tetraline (5.00 g, 28.4 mmol), hydroxylamine hydrochloride (5.92 g, 85.1 mmol) and potassium carbonate 12.94 g, 93.6 mmol) in methanol (100 ml) was heated to reflux and stirred for 16 hrs. The reaction mixture was cooled to room temperature and the solvent was removed under reduced pressure. Water was added to the residue and extraction was carried out with AcOEt. The organic layer was dried over Na₂SO₄— and then concentrated to give 7-methoxy-3,4-dihydro-1 (2H)-naphthalenone oxime (5.51 g, quantitative).
Step 2: To a suspension of Pd/C (10%) in methanol (10 ml) was added a catalytic amount of acetic acid and 7-methoxy-3,4-dihydro-1 (2H)-naphthalenone oxime (2.00 g, 10.5 mmol). The mixture was stirred under a hydrogen atmosphere at room temperature for 16 hrs. The Pd catalyst was removed and the filtrate was concentrated under reduced pressure. Water was added to the residue and extraction was carried out with AcOEt. The organic layer was dried over Na₂SO₄ and then concentrated to give of 7-methoxy-1,2,3,4-tetrahydro-1-naphthalenamine (2.00 g, quantitative).
Step 3: To a solution of 7-methoxy-1,2,3,4-tetrahydro-1-naphthalenamine (0.20 g, 1.1 mmol) in CH₂Cl₂ (5 ml) was added boron tribromide (1.5 ml, 1M solution in CH₂Cl₂) at 0° C. Water was then added to the reaction mixture and extraction was carried out with AcOEt. The organic layer was dried over Na₂SO₄, concentrated under reduced pressure to give 8-amino-5,6,7,8-tetrahydro-2-naphthalenol (0.18 g, 98%).
   [Starting Compound H]

### 1-(3′-amino-1,1′-biphenyl-4-yl)ethanone

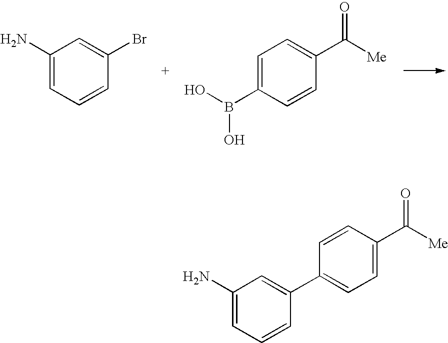

To a stirred solution of 3-bromoaniline (0.344 g, 2.00 mmol) and [Pd(PPh₃)₄] (0.069 g, 0.06 mmol) in DMF was added a 2N solution of sodium carbonate (1.5 ml). 4-acetylphenylboronic acid (0.656 g, 4.00 mmol) was added and the mixture was stirred at 90° C. for 16 hrs. The reaction mixture was then washed with water and dried over MgSO₄. The solution was concentrated under reduced pressure and the resulting residue was purified by preparative thin layer chromatography on silica gel (CHCl₃) to give 1-(3′-amino-1,1′-biphenyl-4-yl)ethanone (0.25 g, 60%).

[Starting compound I]

### 4-amino-1,1′-biphenyl

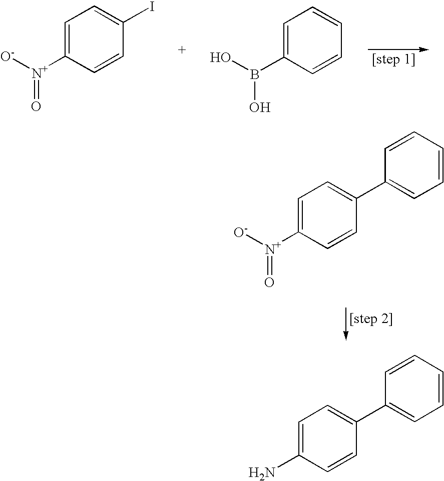 [0188] Step 1: To a stirred mixture of [Pd(PPh₃)₄ (0.069 g, 0.06 mmol), K₃PO₄ (0.636 g, 3.00 mmol) and 4-iodonitrobenzene (0.498 g, 2.00 mmol) in DMF was added phenylboronic acid (0.243 g, 2.00 mmol) and the mixture was stirred at 100° C. for 6 hrs. The reaction mixture was then washed with water and dried over MgSO₄. The solution was concentrated under reduced pressure and the resulting residue was purified by silica gel column chromatography (5% AcOEt-Hexane) to give 4-nitro-1,1′-biphenyl (0.28 g, 69%).
Step 2: To a solution of 4-nitro-1,1′-biphenyl (0.275 g, 1.40 mmol) in ethanol (30 ml) was added Pd/C (0.050 g, 10% with 51.5% water) and the mixture was stirred at room temperature under a hydrogen atmosphere for 5 hrs. The reaction mixture was filtered and the filtrate was concentrated under reduced pressure to give 4-amino-1,1′-biphenyl (0.21 g, 88%).
   [Starting Compound J]

### 3′-methoxy-1,1′-biphenyl-3-amine

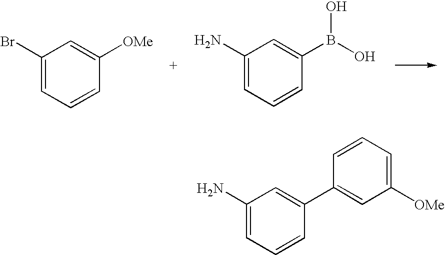

To a stirred solution of 3-bromoanisole (0.374 g, 2.00 mmol) and [Pd(PPh₃)₄] (0.069 g, 0.06 mmol) in DMF was added a 2N solution of sodium carbonate (1.5 ml). 3-aminophenylboronic acid (0.548 g, 4.00 mmol) was added and the mixture was stirred at 90° C. for 16 hrs. The reaction mixture was then washed with water and dried over MgSO₄. The solution was concentrated under reduced pressure and the resulting residue was purified by preparative thin layer chromatography on silica gel (CHCl₃, IPE:Hexane 1:1) to give 3′-methoxy-1,1′-biphenyl-3-amine (0.28 g, 92%).

[Starting Compound K]

### 3-(2-thienyl)aniline

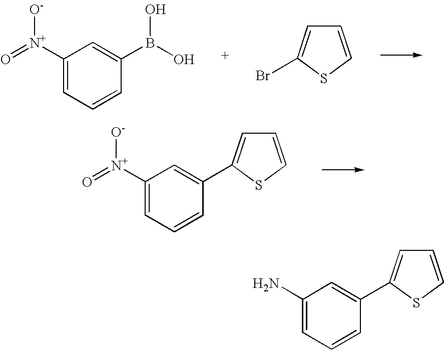

To a stirred mixture of [Pd(PPh₃)₄] (0.069 g, 0.06 mmol), K₃PO₄ (0.636 g, 3.00 mmol) and 2-bromothiophene (0.343 g, 2.00 mmol) in DMF was added 3-nitrophenylboronic acid (0.335 g, 2.00 mmol) and the mixture was stirred at 100° C. for 6 hrs. The reaction mixture was then washed with water and dried over MgSO₄. The solution was concentrated under reduced pressure and the resulting residue was dissolved in ethanol (30 ml). Pd/C (0.050 g, 10% with 51.5% water) was added and the reaction mixture was stirred at room temperature under a hydrogen atmosphere for 5 hrs. The reaction mixture was filtered and the filtrate was concentrated to give 3-(2-thienyl)aniline (0.35 g, 86%).

[Starting Compound L]

### 2-[2-amino-4-(trifluoromethyl)phenyl]ethanol

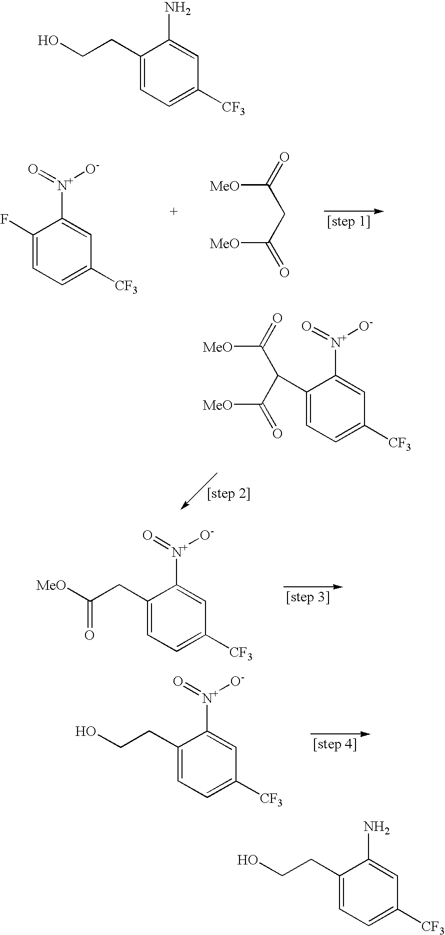 [0195] Step 1: To a suspension of 60% sodium hydride in THF/DMF (30 ml, 1:1) was added dimethyl malonate (2.000 g, 9.57 mmol) at 0° C. The mixture was allowed to warm to room temperature and stirred for another 30 minutes. 4-fluoro-9-3-nitro benzotrifluoride was added and the reaction mixture was stirred for 16 hrs at room temperature. A saturated NH₄Cl solution was added the mixture was extracted with AcOEt. The organic layer was washed with brine and dried over Na₂SO₄. The solution was concentrated under reduced pressure and the resulting residue was purified by silica gel column chromatography (hexane:AcOEt=7:1-3:1) to give dimethyl 2-[2-nitro-4-(trifluoromethyl)phenyl]malonate (1.784 g, 58%).
Step 2: A mixture of 2-[2-nitro-4-(trifluoromethyl)phenyl]malonate (1.780 g, 5.55 mmol), LiCl (0.47 g, 11.11 mmol) in DMSO/water (DMSO 10 ml, water 0.10 ml) was heated to 100° C. and stirred for 5 hrs. After cooling to room temperature, AcOEt was added and the solution was washed with brine. The organic layer was washed with brine and dried over Na₂SO₄ and then concentrated under reduced pressure. The solution was concentrated under reduced pressure and the resulting residue was triturated with ethyl ether/hexane. Collected to give methyl [2-nitro-4-(trifluoromethyl)phenyl]acetate (0.546 g, 37%).
Step 3: To a solution of methyl[2-nitro-4-(trifluoromethyl)phenyl]acetate (0.546 g, 2.07 mmol) in CH₂Cl₂ (25 ml) was added a 0.9M hexane solution of DIBAH (6.90 ml) at −78° C. The reaction temperature was allowed to rise to 0° C. and was stirred for 2 hrs. The reaction was then quenched with iPrOH/H₂O and diluted with AcOEt. SiO₂ was added to the mixture and stirring was continued for another 1 hr. The mixture was passed through a celite pad and the filtrate was concentrated under reduced pressure. The obtained crude residue (0.454 g, 93%) was used in the next step without any further purification.
Step 4: To a solution of 2-[2-nitro-4-(trifluoromethyl)phenyl]ethanol in methanol (20 ml) was added Pd/C (0.050 g, 10%). The solution was stirred at room temperature under a hydrogen atmosphere for 20 hrs. The reaction mixture was filtered, and the filtrate was concentrated under reduced pressure to give 2-[2-amino-4-(trifluoromethyl)phenyl]ethanol. The obtained product was used as starting material without any further purification.
   [Starting Compound M]

### 2-(5-fluoro-2-aminophenyl)ethanol

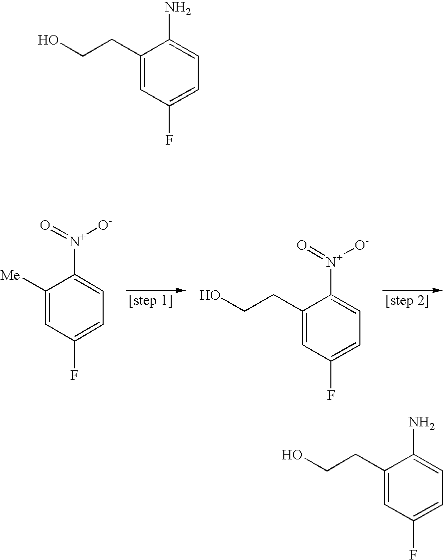 [0200] Step 1: To a stirred mixture of 5-fluoro-2-nitrotoluene (0.300 g, 1.93 mmol) and paraformaldehyde (0.023 g, 0.77 mmol) in DMSO (3.0 ml) was added sodium phenoxide trihydrate (0.010 g, 0.06 mmol). The reacting mixture was heated to 60° C. and stirred for 1 hr. The resulting mixture was diluted with. AcOEt and washed with dil. HCl, water and then brine. The organic layer was dried over Na₂SO₄. The solution was concentrated under reduced pressure and the resulting residue was purified by silica gel column chromatography (hexane:AcOEt 3:1) to give 2-(5-fluoro-2-nitrophenyl)ethanol.
Step 2: A mixture of 2-(5-fluoro-2-nitrophenyl)ethanol (0.123 g, 0.664 mmol), Fe powder (0.300 g, 5.37 mmol) and NH₄Cl (0.100 g, 1.86 mmol) in EtOH/Water (EtOH 8 ml, water 0.4 ml) was stirred at 90° C. for 1 hr. After cooling to room temperature, AcOEt was added and the mixture was filtered through a celite pad. The filtrate was concentrated and the residue was dissolved in AcOEt, washed with water and then brine and dried over MgSO₄. The solution was concentrated under reduced pressure and the resulting residue was purified by silica gel column chromatography (hexane:AcOEt 1:2) to give 2-(5-fluoro-2-aminophenyl)ethanol. (0.09 g, 87%).

Other starting materials are commercially available or can be prepared according to methods reported in the literature.

### Example 1-1

### N-(4-hydroxy-3-methoxybenzyl)-N′-(4-isopropylphenyl)urea

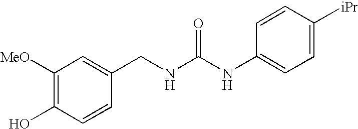

This example was performed according to said method A.

To a stirred solution of 4-(aminomethyl)-2-methoxyphenol hydrochloride (50.0 mg, 0.26 mmol) and triethylamine (26.68 mg, 0.26 mmol) in 1,4-dioxane (1.5 ml) was added a solution of 4-isopropylphenyl isocyanate (38.3 mg, 0.24 mmol) in 1,4-dioxane (1.4 mL) at room temperature. The reaction mixture was warmed to 50° C., and stirred for 20 hrs at the same temperature. The solvent was removed under reduced pressure, and the residue was purified by preparative thin layer chromatography (MeOH:CHCl₃=1:20) to give N-(4-hydroxy-3-methoxybenzyl)-N′-(4-isopropylphenyl)urea (21 mg, 25%).

mp 156° C.;

Molecular weight 314.39

Activity grade: A

### Example 1-2

### N-(3,4-dichlorophenyl)-N′-2-(2-hydroxyethyl)phenyl]urea

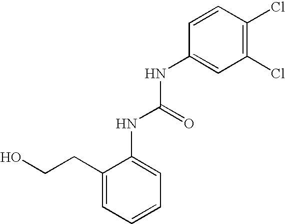

This example was performed according to the general method A.

A solution of 2-(2-aminophenyl)ethanol (30.0 mg, 0.22 mmol) and 3,4-dichlorophenylisocyanate (41.1 mg, 0.22 mmol) in 1,4-dioxane (2.0 mL) was stirred at 50° C. for 18 hrs. The reaction mixture was cooled to room temperature and diluted with diisopropylether. The precipitate was collected and then washed with ⁱPr₂O to give N-(3,4-dichlorophenyl)-N′-[2-(2-hydroxyethyl)phenyl]urea (48.9 mg, 69%).

mp 188-190° C.;

Molecular weight 325.20

Activity grade: A

### Example 2-1

### N-(4′-chloro-1,1′-biphenyl-3-yl)-N′-(4-hydroxy-3 methoxybenzyl)urea

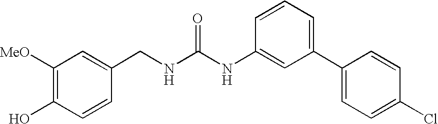

This example was performed according to said method B.

A mixture of 4-(aminomethyl)-2-methoxyphenol hydrochloride (50.0 mg, 0.26 mmol) and phenyl 4′-chloro-1,1′-biphenyl-3-ylcarbamate (85.4 mg, 0.26 mmol) in DMSO (0.5 ml) was heated to 90° C. and stirred for 16 hrs. Water was then added and extraction was carried out with AcOEt. The organic layer was dried over Na₂SO₄ and then concentrated under reduced pressure. The residue was purified by silica gel column chromatography (AcOEt:hexane=2:3) to give N-(4′-chloro-1,1′-biphenyl-3-yl)-N′-(4-hydroxy-3 methoxybenzyl)urea (65.0 mg, 64%).

m.p. 153.4° C.

Molecular weight 382.85

Activity grade: A

### Example 2-2

### N-[2-(2-hydroxyethyl)phenyl]-N′-[3(trifluoromethoxy)phenyl]urea

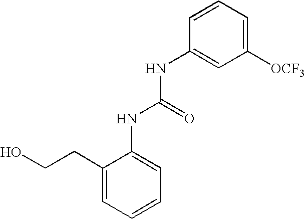

This example was performed according to the general method B.

A solution of 2-(2-aminophenyl)ethanol (80.1 mg, 0.58 mmol) and phenyl 3-(trifluoromethoxy)phenylcarbamate (165.3 mg, 0.56 mmol) in DMSO (2.0 mL) was stirred at 90° C. for 1 hr. The reaction mixture was cooled to room temperature and diluted with AcOEt. The solution was washed with 1N HCl, 1N NaOH and brine dried over Na₂SO₄. The solution was then concentrated under reduced pressure, and the residue was triturated with diisopropylether to give N-[2-(2-hydroxyethyl)phenyl]-N′-[3(trifluoromethoxy)phenyl]urea (70.5 mg, 37%).

mp 160-161° C.;

Molecular weight 340.30

Activity grade: A

### Example 3-1

### N-(1,1′-biphenyl-3-yl)-N′-[2-(2-hydroxyethyl)phenyl]urea

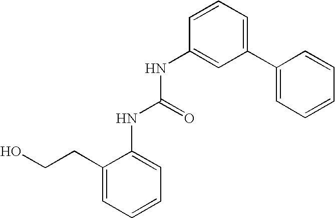

This example was performed according to the general method C.

To a solution of 1,1′-biphenyl-3-amine (37.0 mg, 0.22 mmol) in THF (2.0 ml) was added 1′-carbonyldi(1,2,4-triazole) (35.9 mg, 0.22 mmol). 2-(2-aminophenyl)ethanol (30.0 mg, 0.22 mmol) was added and the mixture was stirred at 55° C. for 18 hrs. After cooling to room temperature, the mixture was diluted with water and ethylalcohol and the resulting precipitate was collected and washed to give N-(1,1′-biphenyl-3-yl)-N′-[2-(2-hydroxyethyl)phenyl]urea (20.8 mg, 29%).

mp 196-198° C.

Molecular weight 332.41

MS (M+H): 333

Activity grade: A

According to procedures similar to any one of the Examples 1 to 3 above, the following compounds were synthesized and tested.

TABLE 1

| | | | | melting | hVR1 |
|---|---|---|---|---|---|
| Ex. No | MOLSTRUCTURE | MW | M + 1 | point | class |
| 6 | 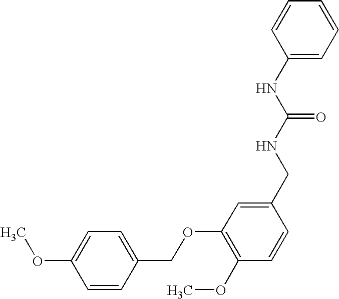 | 392,45873 | 393 | ND | C |
| 7 | 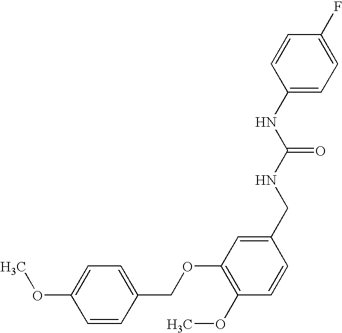 | 410,44916 | 411 | ND | C |
| 8 | 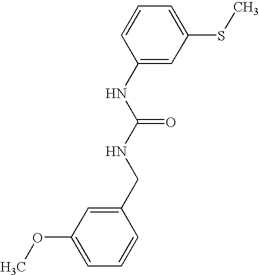 | 302,39806 | 303 | ND | C |
| 9 | 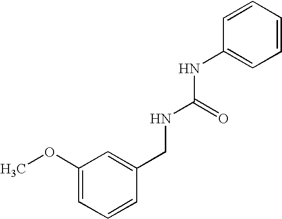 | 256,30697 | 257 | ND | C |
| 10 | 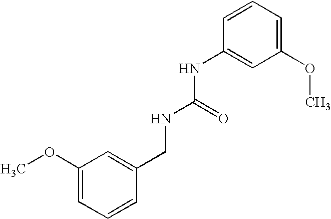 | 286,33346 | 287 | ND | C |
| 11 | 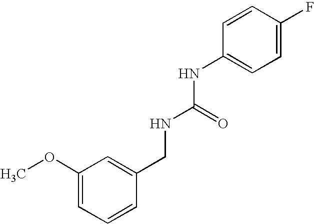 | 274,2974 | 275 | ND | C |
| 12 | 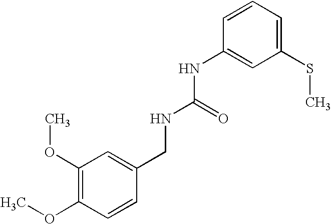 | 332,42455 | 333 | ND | C |
| 13 | 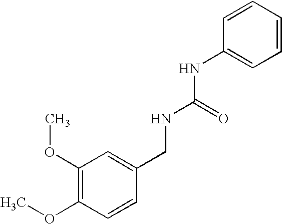 | 286,33346 | 287 | ND | C |
| 14 | 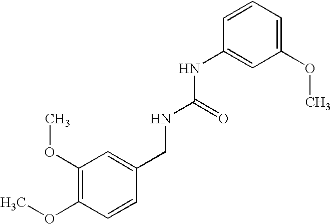 | 316,35995 | 317 | ND | C |
| 15 | 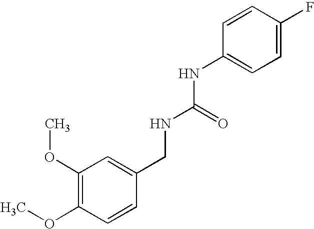 | 304,32389 | 305 | ND | C |
| 16 | 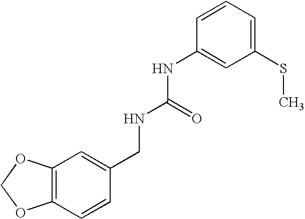 | 316,38152 | 317 | ND | C |
| 17 | 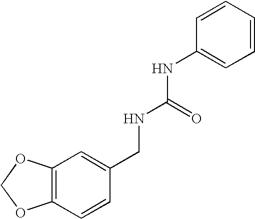 | 270,29043 | 271 | ND | C |
| 18 | 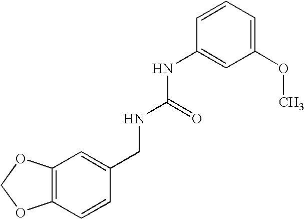 | 300,31692 | 301 | ND | C |
| 19 | 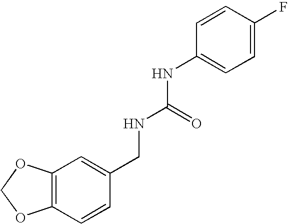 | 288,28086 | 289 | ND | C |
| 20 | 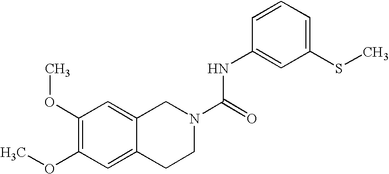 | 358,46279 | 359 | ND | C |
| 21 | 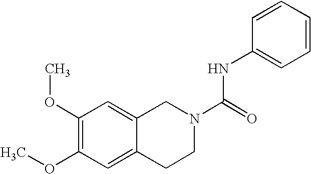 | 312,3717 | 313 | ND | C |
| 22 | 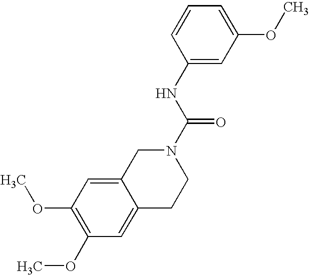 | 342,39819 | 343 | ND | C |
| 23 | 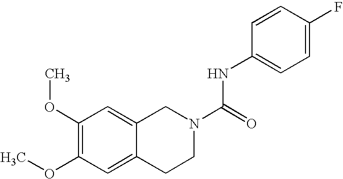 | 330,36213 | 331 | ND | C |
| 24 | 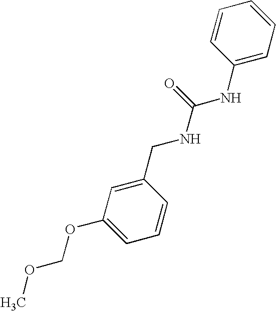 | 286,33346 | 287 | ND | C |
| 25 | 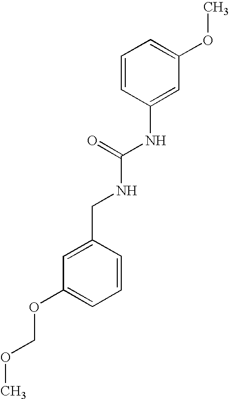 | 316,35995 | 317 | ND | C |
| 26 | 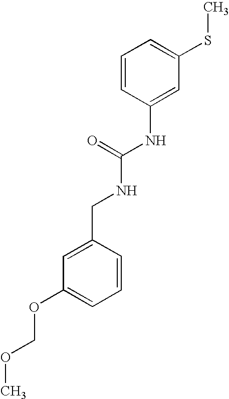 | 332,42455 | 333 | ND | C |
| 27 | 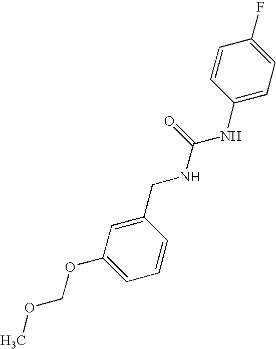 | 304,32389 | 305 | ND | C |
| 28 | 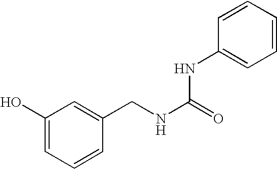 | 242,27988 | 243 | ND | C |
| 29 | 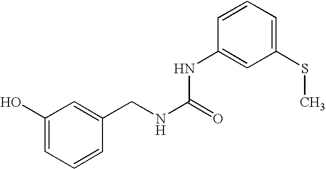 | 288,37097 | 289 | ND | C |
| 30 | 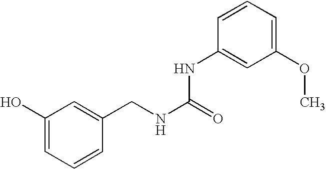 | 272,30637 | 273 | ND | C |
| 31 | 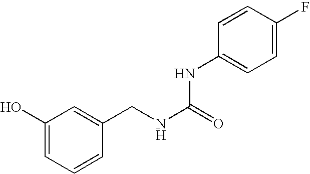 | 260,27031 | 261 | ND | C |
| 32 | 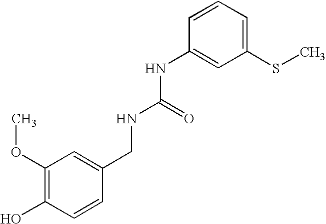 | 318,39746 | 319 | ND | B |
| 33 | 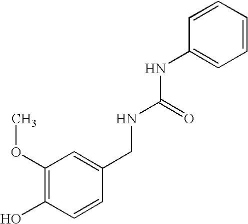 | 272,30637 | 273 | ND | C |
| 34 | 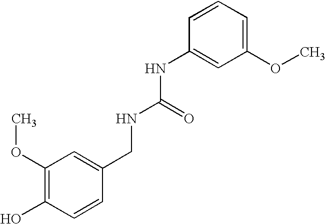 | 302,33286 | 303 | 133 | C |
| 35 | 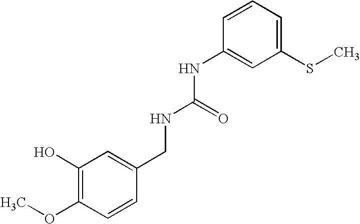 | 318,39746 | 319 | ND | c |
| 36 | 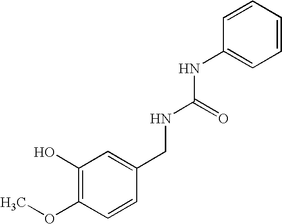 | 272,30637 | 273 | ND | C |
| 37 | 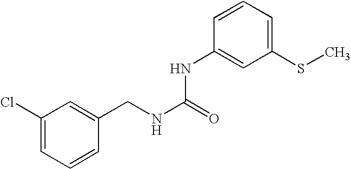 | 306,8166 | 307 | ND | C |
| 38 | 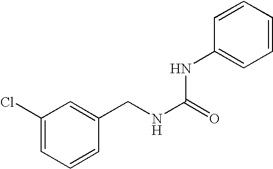 | 260,72551 | 261 | ND | C |
| 39 | 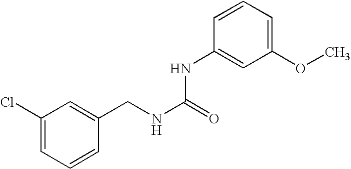 | 290,752 | 291 | ND | C |
| 40 | 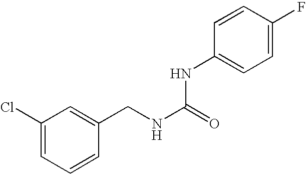 | 278,71594 | 279 | ND | C |
| 41 | 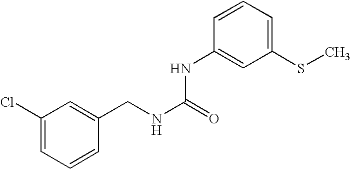 | 341,26163 | 341 | ND | C |
| 42 | 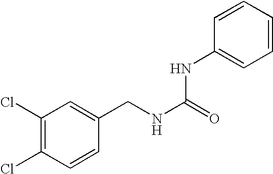 | 295,17054 | 295 | ND | B |
| 43 | 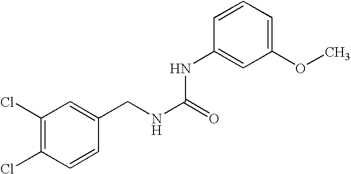 | 325,19703 | 325 | ND | B |
| 44 | 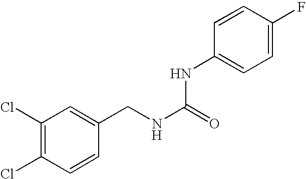 | 313,16097 | 313 | ND | B |
| 45 | 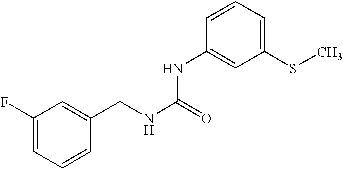 | 290,362 | 291 | ND | C |
| 46 | 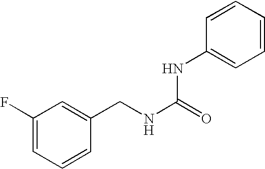 | 244,27091 | 245 | ND | C |
| 47 | 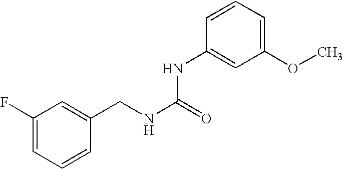 | 274,2974 | 275 | ND | C |
| 48 | 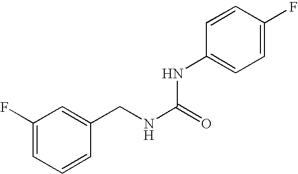 | 262,26134 | 263 | ND | C |
| 49 | 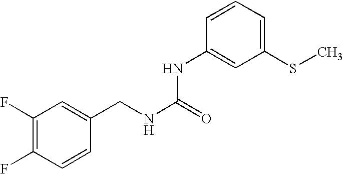 | 308,35243 | 309 | ND | C |
| 50 | 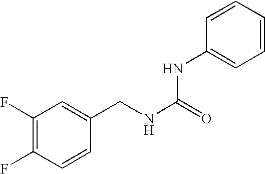 | 262,26134 | 263 | ND | C |
| 51 | 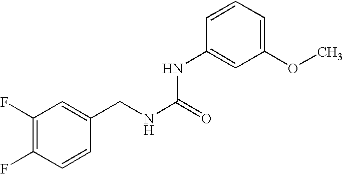 | 292,28783 | 293 | ND | C |
| 52 | 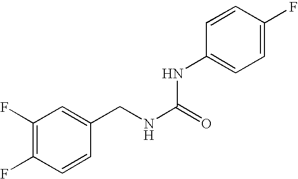 | 280,25177 | 281 | ND | C |
| 53 | 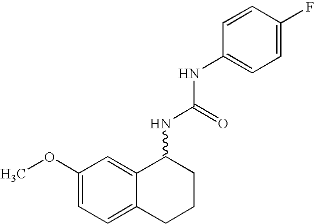 | 314,36273 | | 177.5 | C |
| 54 | 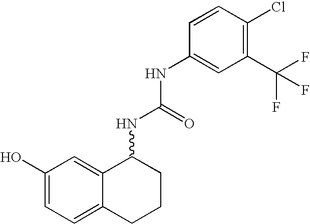 | 384,78862 | | 201 | B |
| 55 | 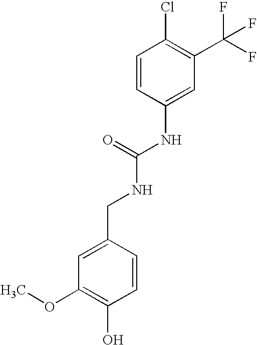 | 374,74978 | | 193 | A |
| 56 | 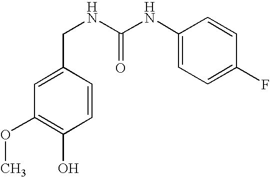 | 290,2968 | | 211-212 | C |
| 57 | 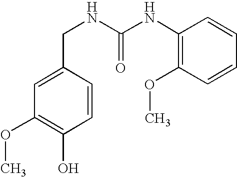 | 302,33286 | | 144 | C |
| 58 | 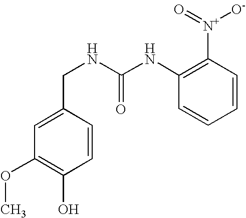 | 317,3039 | | 180 | C |
| 59 | 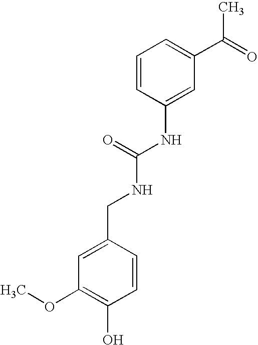 | 314,34401 | 315 | ND | C |
| 60 | 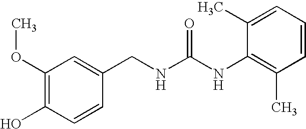 | 300,36055 | | 211 | C |
| 61 | 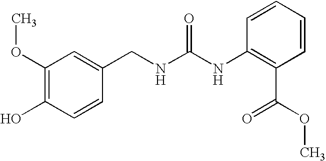 | 330,34341 | 331 | ND | C |
| 62 | 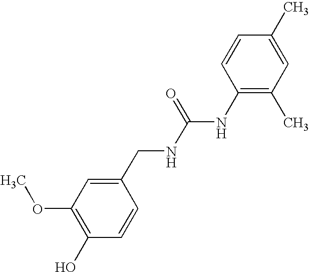 | 300,36055 | | 184 | C |
| 63 | 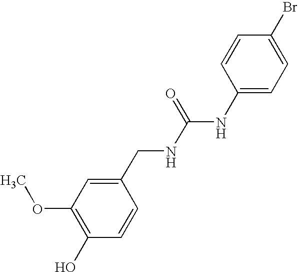 | 351,2024 | | 203 | B |
| 64 | 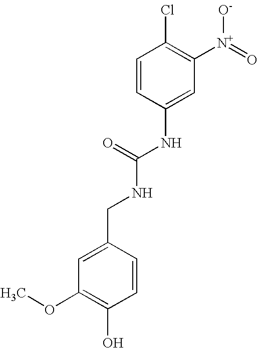 | 351,74893 | | 180 | B |
| 65 | 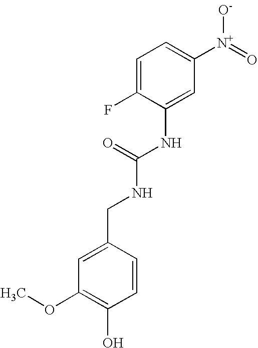 | 335,29433 | | 208 | c |
| 66 | 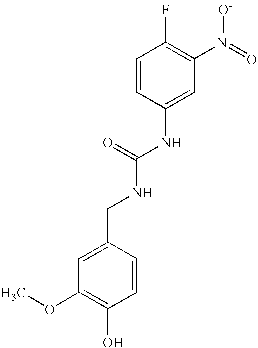 | 335,29433 | | 184 | B |
| 67 | 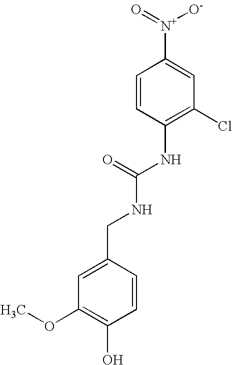 | 351,74893 | | 195Z | C |
| 68 | 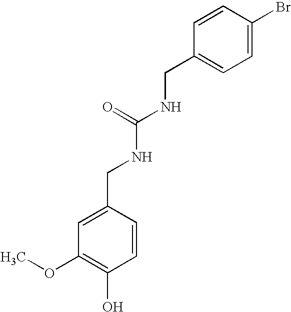 | 365,22949 | | 195Z | B |
| 69 | 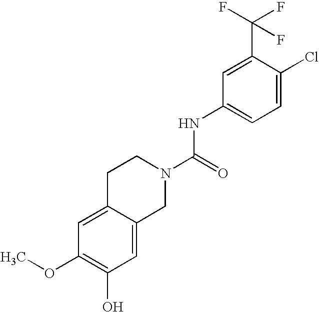 | 400,78802 | 401 | ND | B |
| 70 | 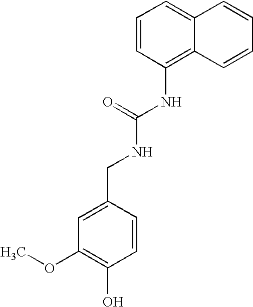 | 322,36691 | | 149 | A |
| 71 | 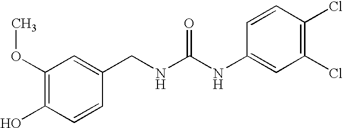 | 341,19643 | | 207 | A |
| 72 | 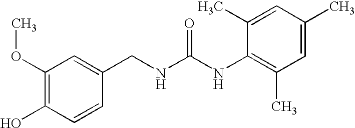 | 314,38764 | | 217 | C |
| 73 | 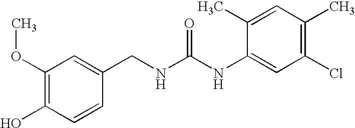 | 320,77849 | 321 | ND | A |
| 74 | 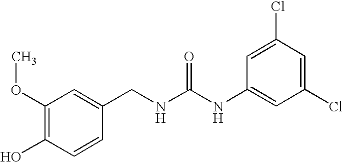 | 341,19643 | 342 | ND | ? |
| 75 | 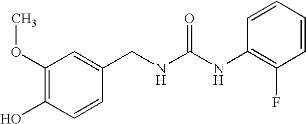 | 290,2968 | 291 | ND | B |
| 76 | 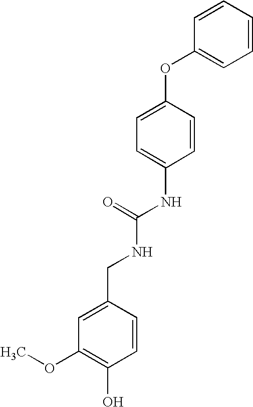 | 364,40455 | 364 | ND | A |
| 77 | 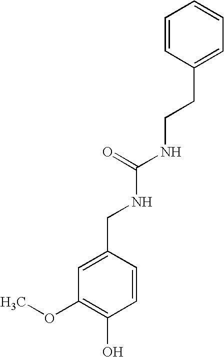 | 300,36055 | 301 | ND | B |
| 78 | 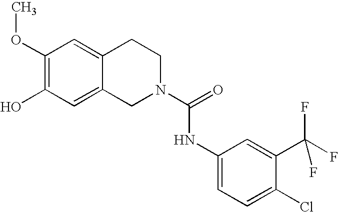 | 400,78802 | | 130 | B |
| 79 | 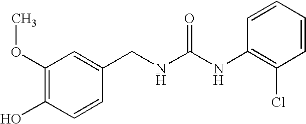 | 306,7514 | 306 | ND | B |
| 80 | 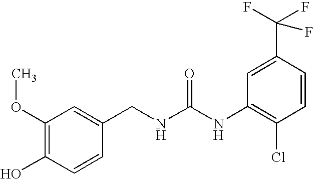 | 374,74978 | 375 | ND | A |
| 81 | 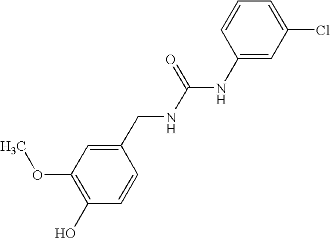 | 306,7514 | 307 | ND | A |
| 82 | 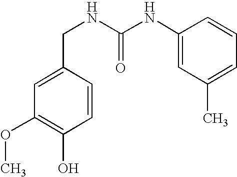 | 286,33346 | | 181 | B |
| 83 | 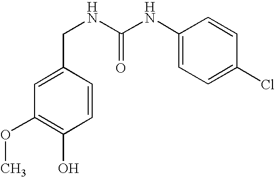 | 306,7514 | | 210 | A |
| 84 | 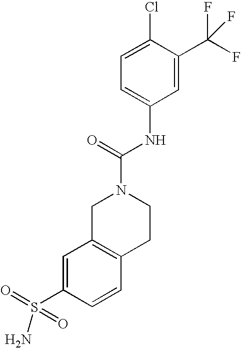 | 433,8396 | ND | ND | B |
| 85 | 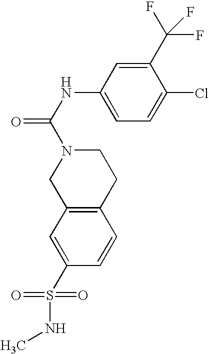 | 447,86669 | 448 | ND | C |
| 86 | 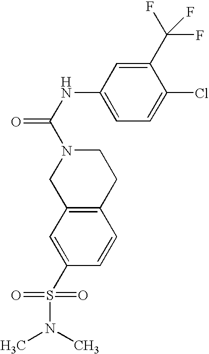 | 461,89378 | ND | ND | C |
| 87 | 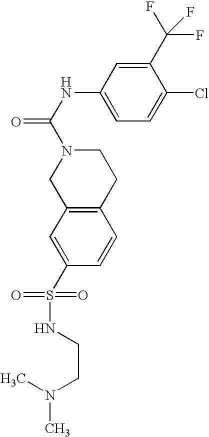 | 504,96263 | 505 | ND | C |
| 88 | 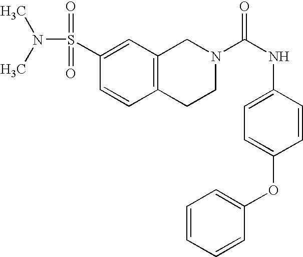 | 451,54855 | 452 | ND | C |
| 89 | 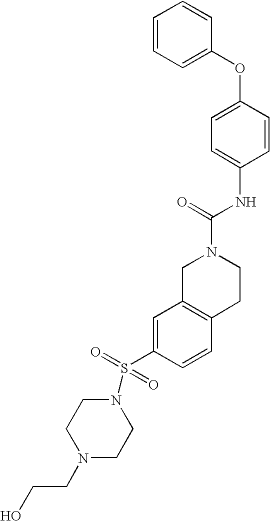 | 536,65504 | 537 | ND | C |
| 90 | 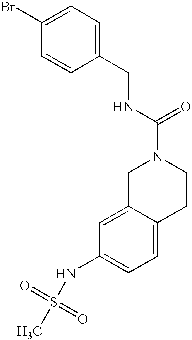 | 438,3464 | 439 | ND | C |
| 91 | 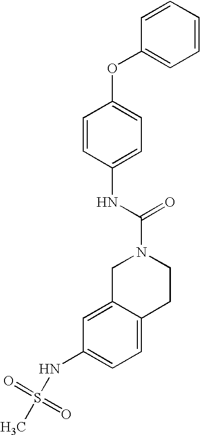 | 437,52146 | 438 | ND | C |
| 92 | 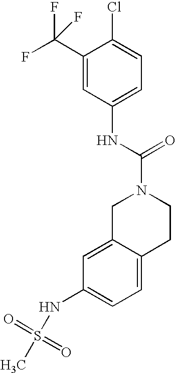 | 447,86669 | 448 | ND | B |
| 93 | 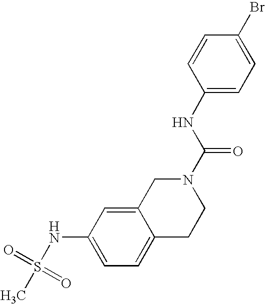 | 424,31931 | 425 | ND | C |
| 94 | 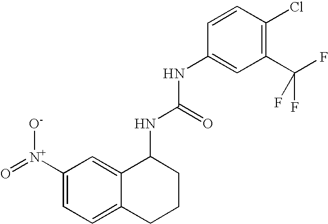 | 413,78675 | 414 | ND | C |
| 95 | 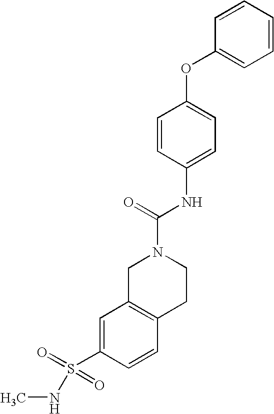 | 437,52146 | ND | ND | C |
| 96 | 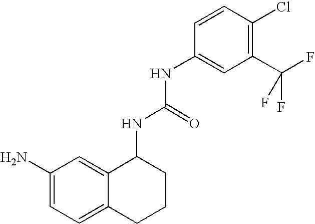 | 383,80389 | | 178 | C |
| 97 | 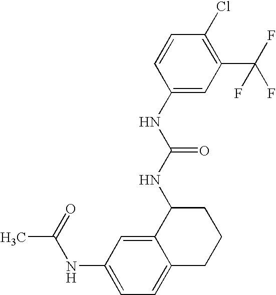 | 425,84153 | 426 | ND | C |
| 98 | 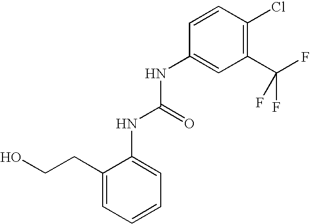 | 358,75038 | | 175-177 | A |
| 99 | 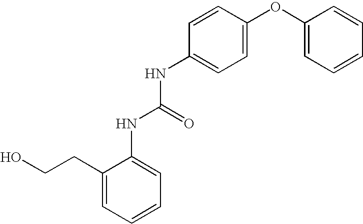 | 348,40515 | | 133-135 | A |
| 100 | 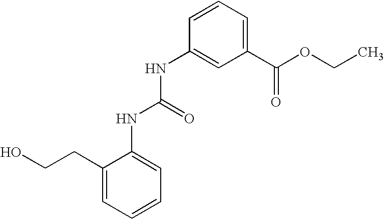 | 325,3711 | | 152-153 | B |
| 101 | 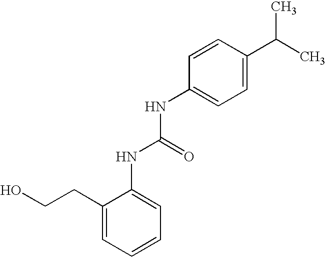 | 298,38824 | | 149-150 | B |
| 102 | 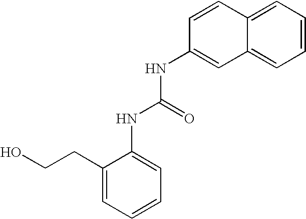 | 306,36751 | | 195-197 | B |
| 103 | 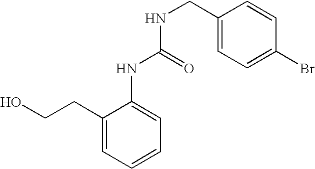 | 349,23009 | | 198-200 | B |
| 104 | 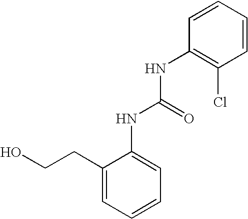 | 290,752 | | 173-175 | C |
| 105 | 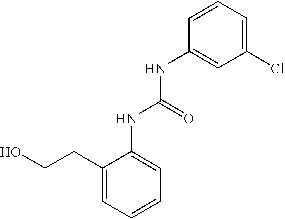 | 290,752 | | 188-190 | C |
| 106 | 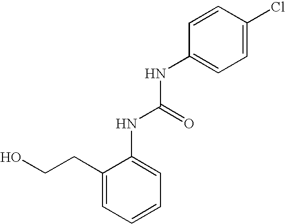 | 290,752 | | 185-187 | C |
| 107 | 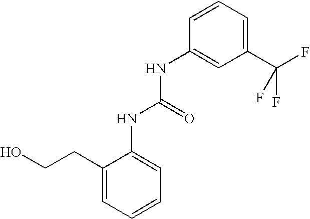 | 324,30535 | | 173-175 | B |
| 108 | 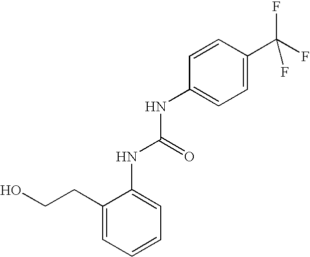 | 324,30535 | | 178-180 | B |
| 109 | 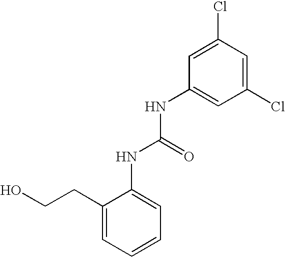 | 325,19703 | | 214-216 | B |
| 110 | 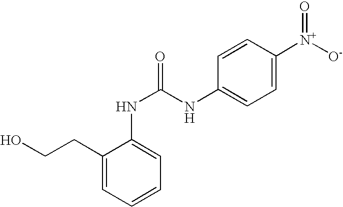 | 335,74953 | | 178 | C |
| 111 | 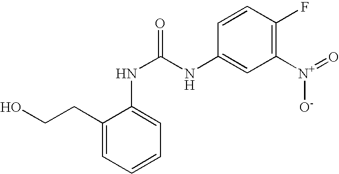 | 319,29493 | | 185 | C |
| 112 | 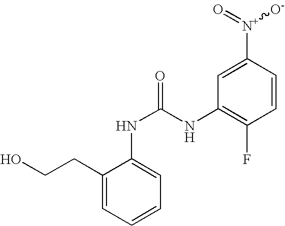 | 319,29493 | | 183 | C |
| 113 | 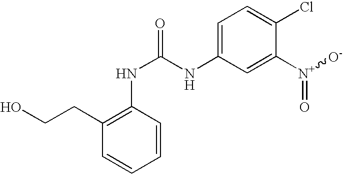 | 335,74953 | | 170 | B |
| 114 | 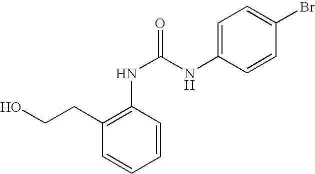 | 335,203 | | 208 | C |
| 115 | 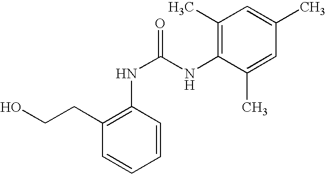 | 298,38824 | | 243 | C |
| 116 | 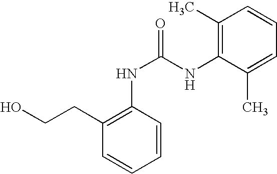 | 284,36115 | | 226 | C |
| 117 | 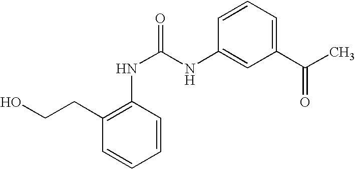 | 298,34461 | | 177 | C |
| 118 | 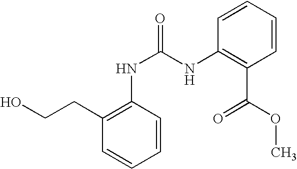 | 314,34401 | | 128 | C |
| 119 | 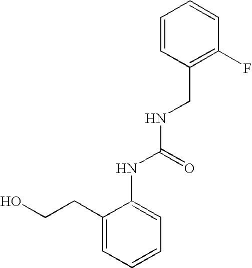 | 288,32449 | | 141-143 | C |
| 120 | 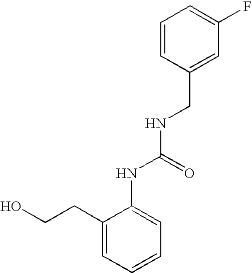 | 288,32449 | | 160-162 | C |
| 121 | 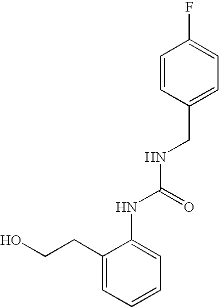 | 288,32449 | | 165-167 | C |
| 122 | 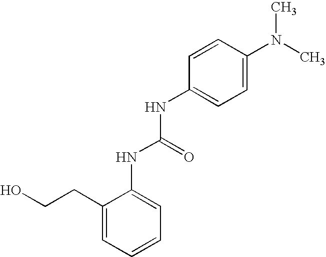 | 299,37582 | | 187-188 | C |
| 123 | 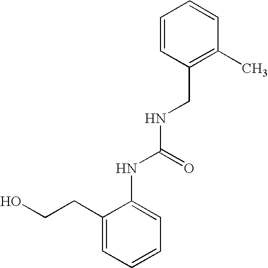 | 284,36115 | | 186-188 | C |
| 124 | 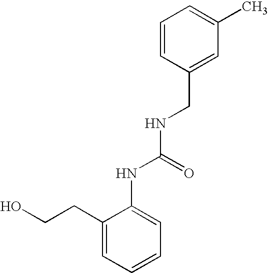 | 284,36115 | | 148-150 | C |

| | | | | melting | hVR1 |
|---|---|---|---|---|---|
| Ex. No | MOLSTRUCTURE | MW | M + 1 | point | class |
| 125 | 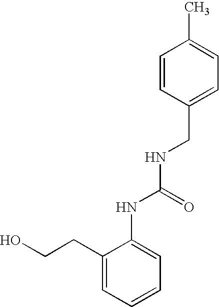 | 284,36115 | | 181-183 | C |
| 126 | 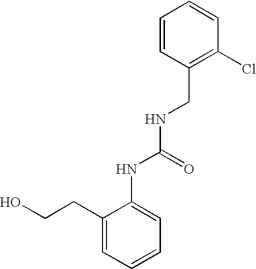 | 304,77909 | | 183-185 | C |
| 127 | 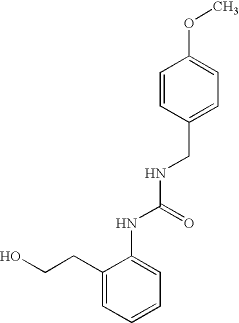 | 300,36055 | | 175-177 | C |
| 128 | 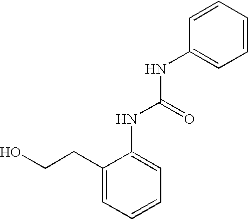 | 256,30697 | 385 | ND | C |
| 129 | 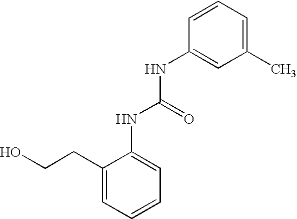 | 270,33406 | | 186 | C |
| 130 | 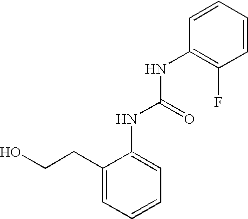 | 274,2974 | 275 | ND | C |
| 131 | 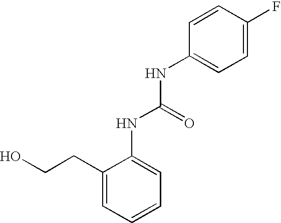 | 274,2974 | | 183 | C |
| 132 | 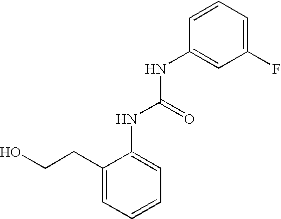 | 274,2974 | | 166 | C |
| 133 | 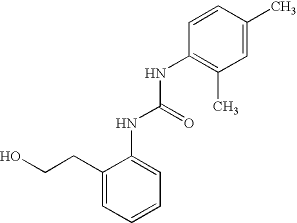 | 284,36115 | | 181 | C |
| 134 | 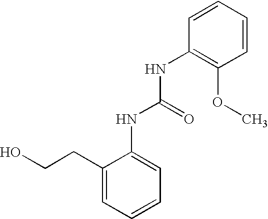 | 286,33346 | | 154 | C |
| 135 | 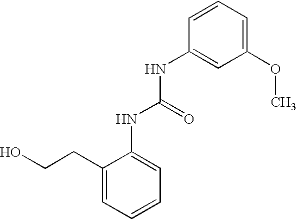 | 286,33346 | | 169 | C |
| 136 | 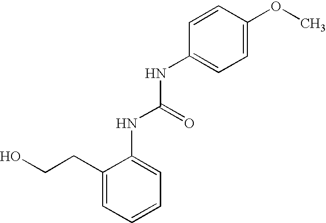 | 286,33346 | | 186 | C |
| 137 | 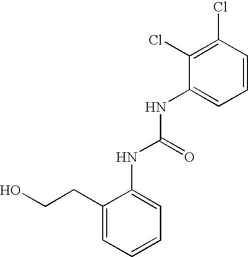 | 325,19703 | 326 | ND | C |
| 138 | 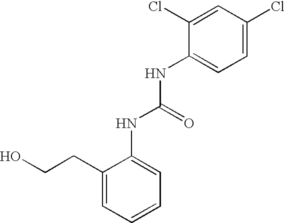 | 325,19703 | 326 | ND | C |
| 139 | 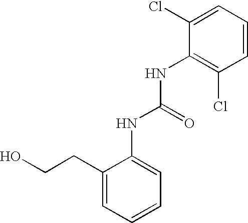 | 325,19703 | 326 | ND | C |
| 140 | 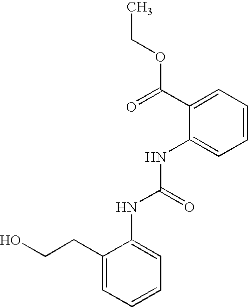 | 328,3711 | 329 | ND | C |
| 141 | 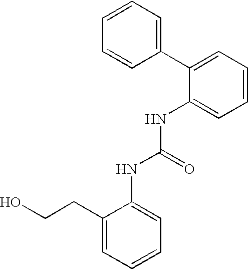 | 332,40575 | 333 | ND | C |
| 142 | 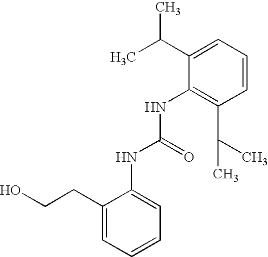 | 340,46951 | 344 | ND | C |
| 143 | 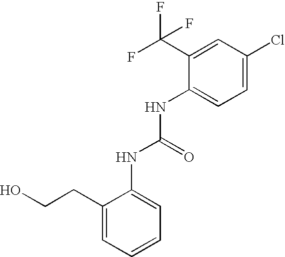 | 358,75038 | 359 | ND | C |
| 144 | 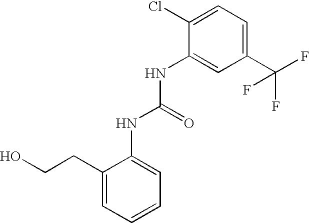 | 358,75038 | 359 | ND | B |
| 145 | 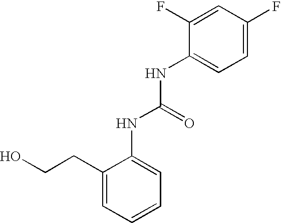 | 292,28783 | | 174-176Z | C |
| 146 | 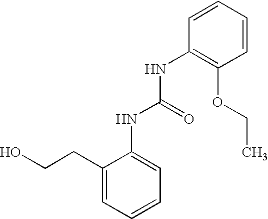 | 300,36055 | | 112-114Z | C |
| 147 | 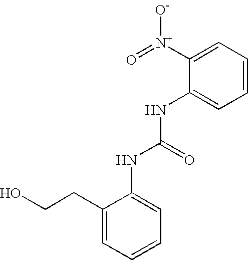 | 301,3045 | | 112-114 | C |
| 148 | 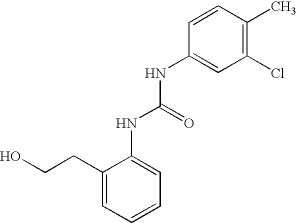 | 304,77909 | | 195-196 | C |
| 149 | 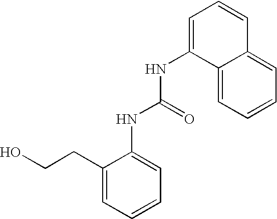 | 306,36751 | | 188-191Z | C |
| 150 | 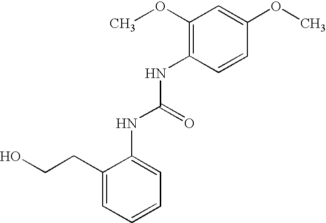 | 316,35995 | | 189-190 | C |
| 151 | 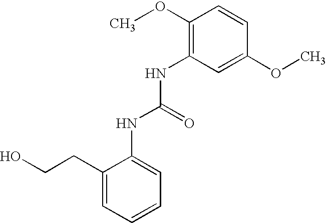 | 316,35995 | | 157-158 | C |
| 152 | 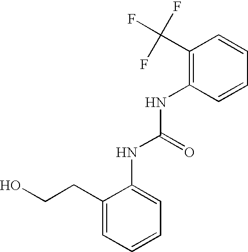 | 324,30535 | | 180-182 | C |
| 153 | 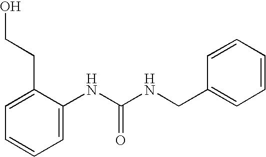 | 270,33406 | | 149-150 | C |
| 154 | 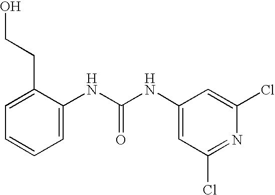 | 326,18461 | | 194 | C |
| 155 | 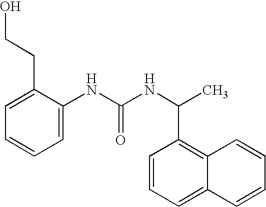 | 334,42169 | | 176 | C |
| 156 | 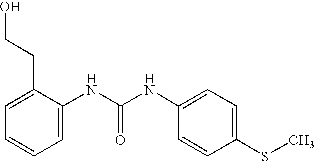 | 302,39806 | | 176-177 | C |
| 157 | 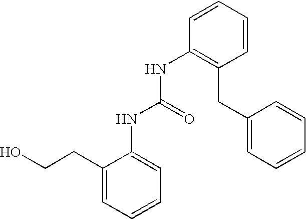 | 346,43284 | | 162-164 | C |
| 158 | 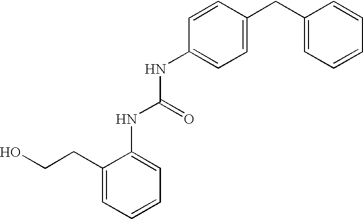 | 346,43284 | | 164-166 | B |
| 159 | 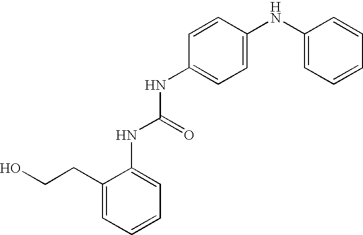 | 347,42042 | | 150-151 | C |
| 160 | 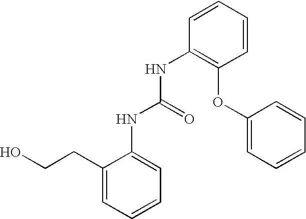 | 348,40515 | | 190-191 | C |
| 161 | 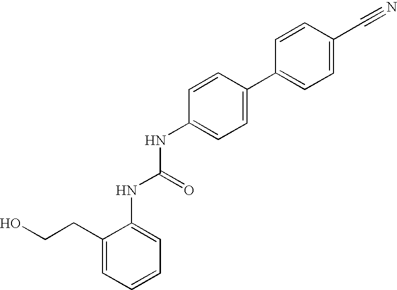 | 357,41563 | | 213-215 | C |
| 162 | 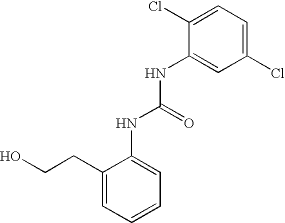 | 325,19703 | 326 | ND | C |
| 163 | 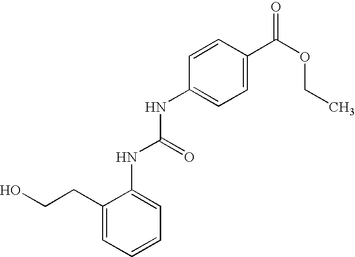 | 328,3711 | 326 | ND | C |
| 164 | 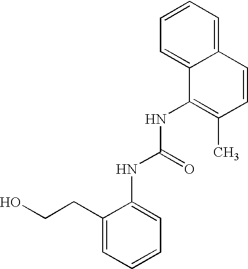 | 320,3946 | | 204 | C |
| 165 | 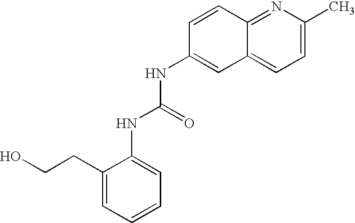 | 321,38218 | | 207 | C |
| 166 | 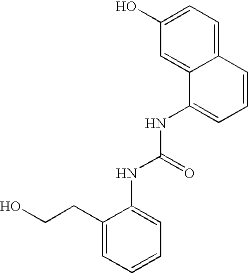 | 322,36691 | | 212 | C |
| 167 | 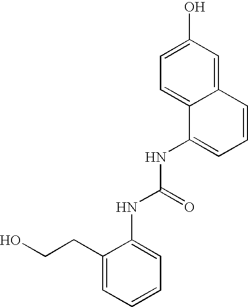 | 322,36691 | | 188Z | C |
| 168 | 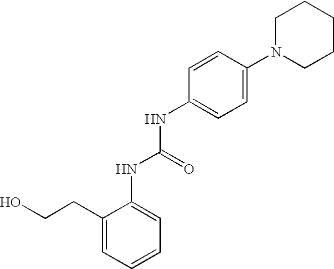 | 339,44115 | | 188 | C |
| 169 | 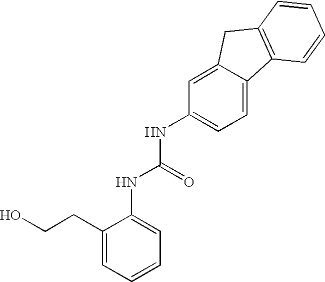 | 344,4169 | | >250 | A |
| 170 | 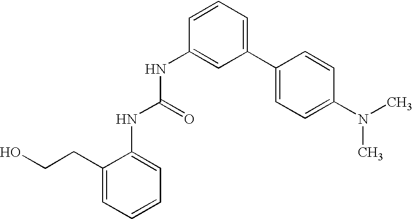 | 375,4746 | 326 | ND | A |
| 171 | 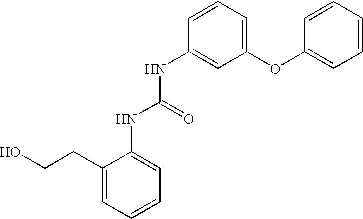 | 348,40515 | | 145-146 | A |
| 172 | 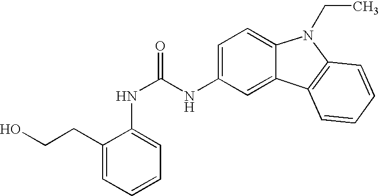 | 373,45866 | | 224-226 | A |
| 173 | 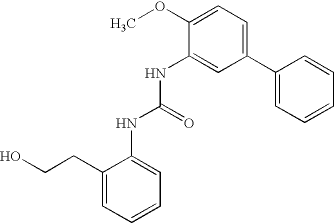 | 362,43224 | | 178-180 | B |
| 174 | 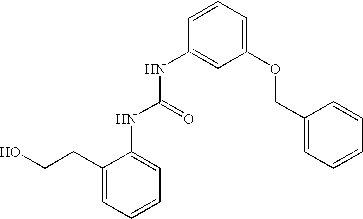 | 362,43224 | | 146-148 | A |
| 175 | 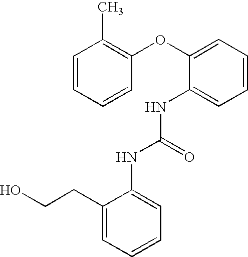 | 362,43224 | | 170-172 | C |
| 176 | 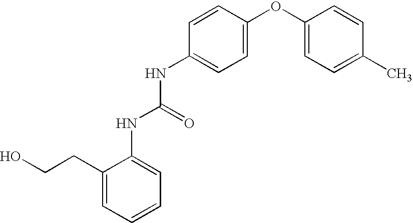 | 362,43224 | | 164-168Z | A |
| 177 | 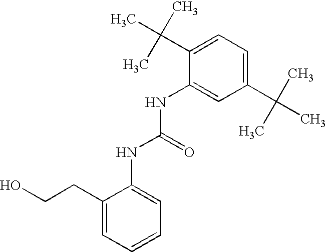 | 368,52369 | 369 | ND | C |
| 178 | 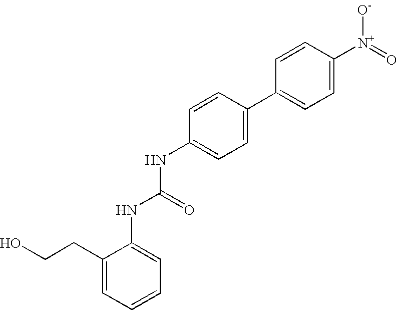 | 377,40328 | 378 | ND | B |
| 179 | 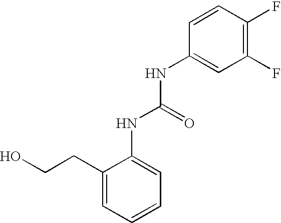 | 292,28783 | | 162-164 | C |
| 180 | 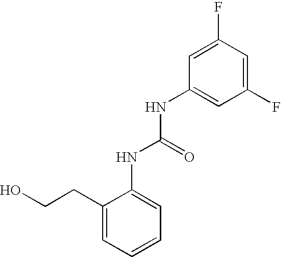 | 292,28783 | | 188-189 | B |
| 181 | 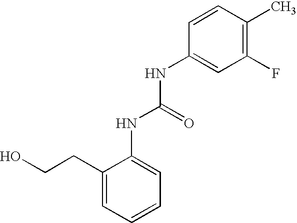 | 288,32449 | | 184-186 | C |
| 182 | 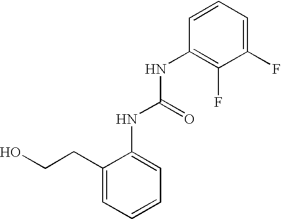 | 292,28783 | | 159-161 | C |
| 183 | 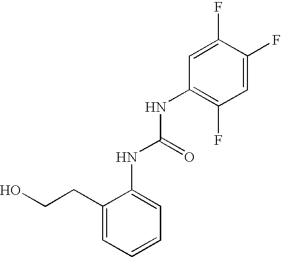 | 310,27826 | | 172-174 | C |
| 184 | 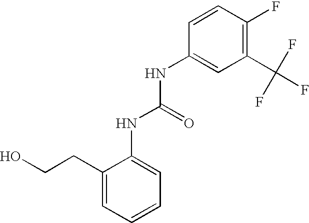 | 342,29578 | | 158-161Z | A |
| 185 | 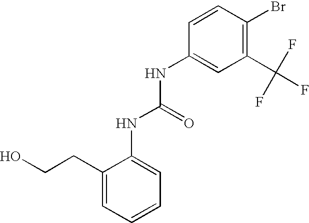 | 403,20138 | | 161-164 | A |
| 186 | 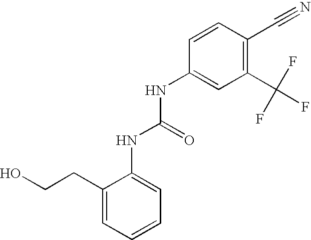 | 349,31523 | | 171 | A |
| 187 | 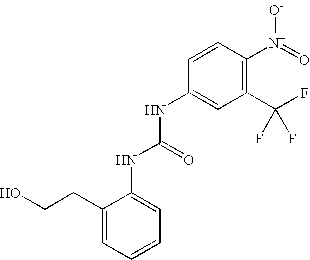 | 369,30288 | | 149-150 | B |
| 188 | 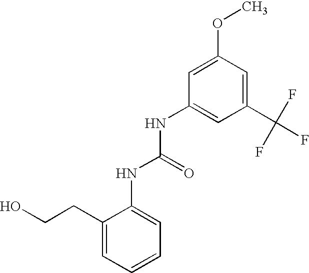 | 354,33184 | | 161-162 | A |
| 189 | 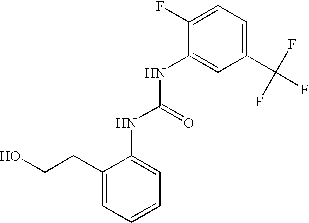 | 342,29578 | | 150-152 | C |
| 190 | 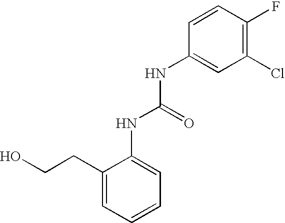 | 308,74243 | | 193-194 | B |
| 191 | 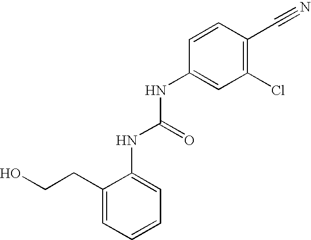 | 315,76188 | | 186-187 | B |
| 192 | 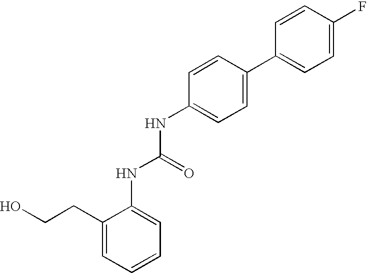 | 350,39618 | | 197 | B |
| 193 | 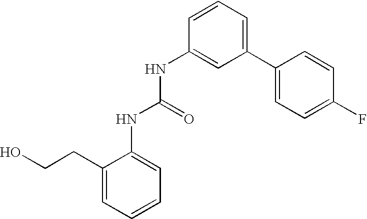 | 350,39618 | 351 | ND | A |
| 194 | 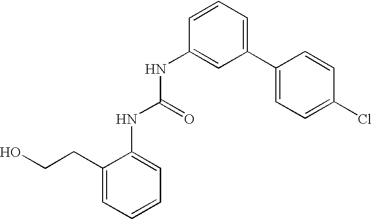 | 366,85078 | 367 | ND | A |
| 195 | 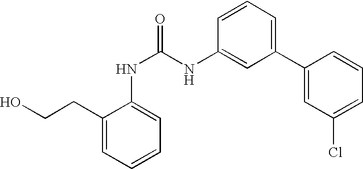 | 366,85078 | | 175 | A |
| 196 | 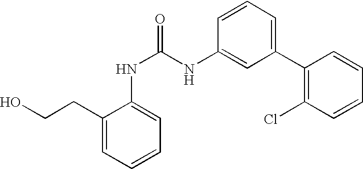 | 366,85078 | | 153 | A |
| 197 | 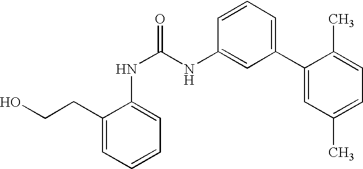 | 360,45993 | | 167 | A |
| 198 | 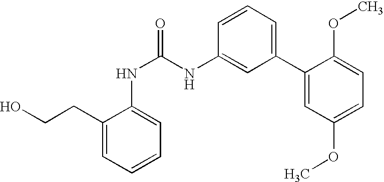 | 392,45873 | | 170 | B |
| 199 | 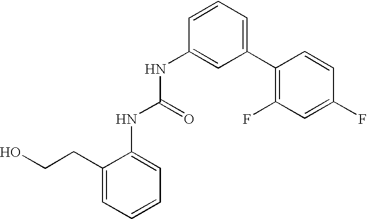 | 368,38661 | | 169 | A |
| 200 | 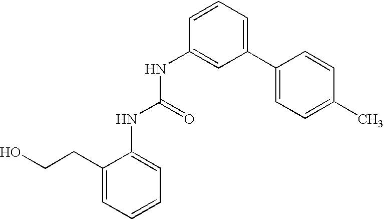 | 346,43284 | | 178 | A |
| 201 | 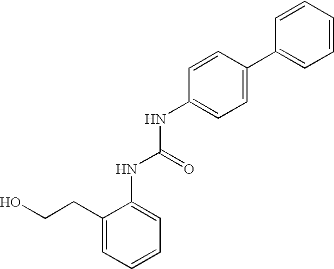 | 332,40575 | | 194 | B |
| 202 | 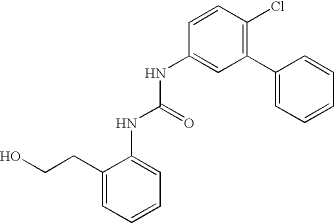 | 366,85078 | | 185 | B |
| 203 | 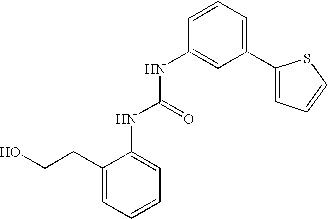 | 338,43151 | | 195 | A |
| 204 | 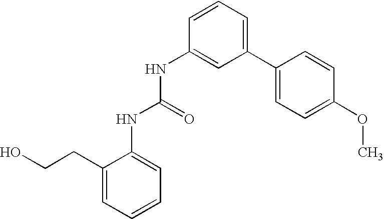 | 362,43224 | | 166 | A |
| 205 | 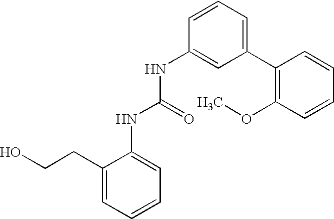 | 362,43224 | | 130 | B |
| 206 | 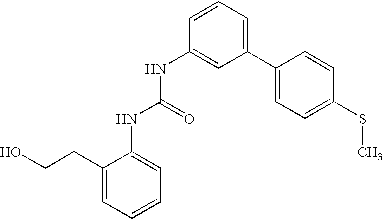 | 378,49684 | 379 | 191 | A |
| 207 | 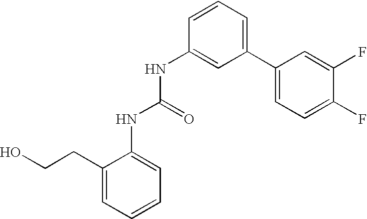 | 368,38661 | 369 | 181 | A |
| 208 | 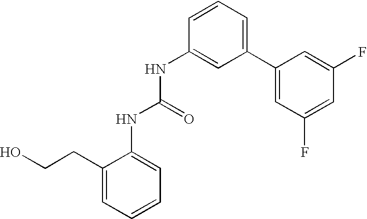 | 368,38661 | 369 | 169 | A |
| 209 | 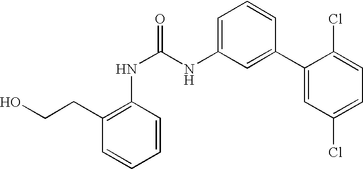 | 401,29581 | | 142 | A |
| 210 | 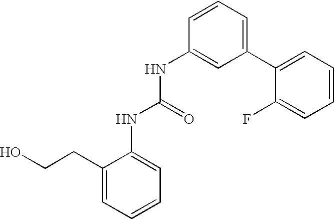 | 350,39618 | | 171-172 | A |
| 211 | 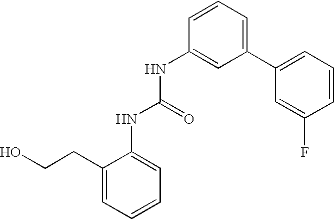 | 350,39618 | | 188 | A |
| 212 | 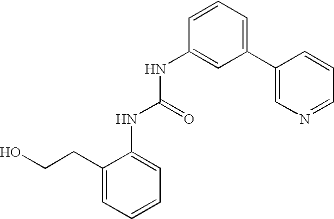 | 333,39333 | | 178.9 | C |
| 213 | 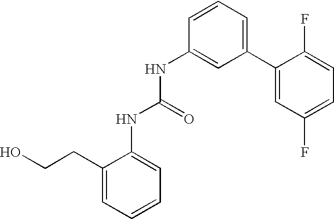 | 368,38661 | 369 | 150 | A |
| 214 | 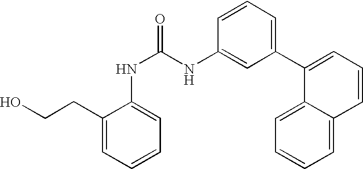 | 382,46629 | | 113 | A |
| 215 | 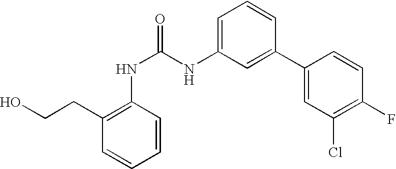 | 384,84121 | | 176 | A |
| 216 | 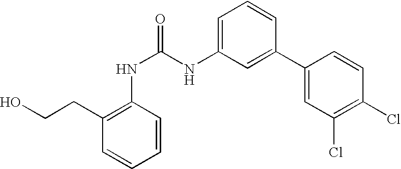 | 401,29581 | | 180 | A |
| 217 | 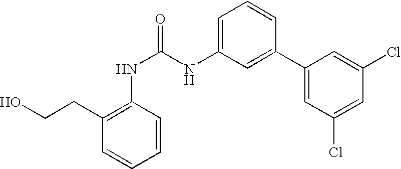 | 401,29581 | | 184 | A |
| 218 | 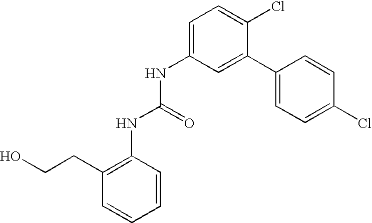 | 401,29581 | | 170-172 | A |
| 219 | 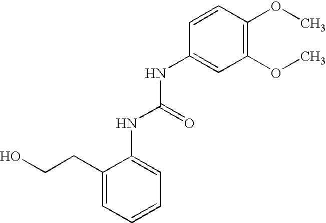 | 316,35995 | | 186-187 | C |
| 220 | 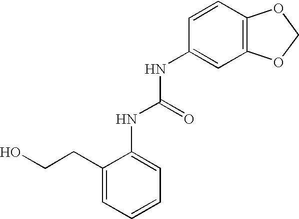 | 300,31692 | | 183-184 | C |
| 221 | 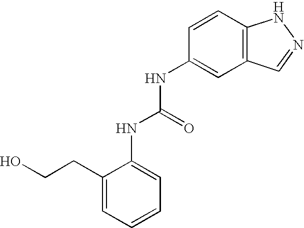 | 296,33152 | | 234-236 | C |
| 222 | 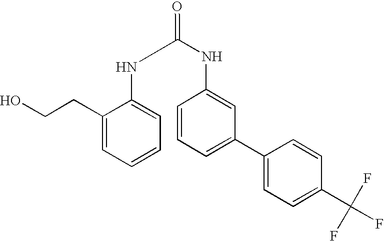 | 400,40413 | | 198 | A |
| 223 | 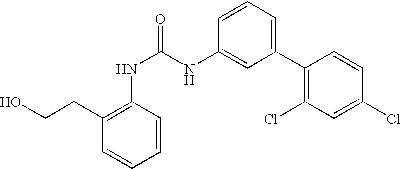 | 401,29581 | | 166 | A |
| 224 | 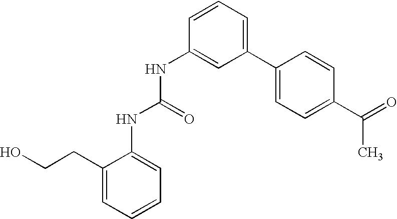 | 374,44339 | | 205 | A |
| 225 | 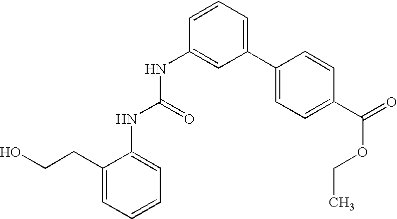 | 404,46988 | | 196 | A |
| 226 | 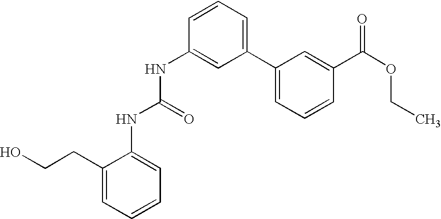 | 404,46988 | | 133 | A |
| 227 | 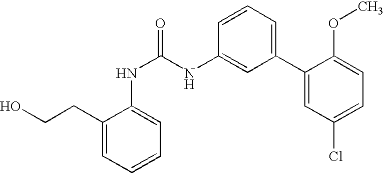 | 392,45873 | | 141-143 | A |
| 228 | 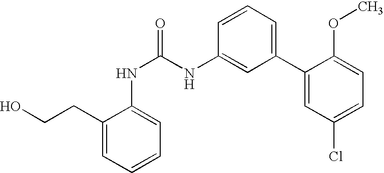 | 396,87727 | | 126-129 | A |
| 229 | 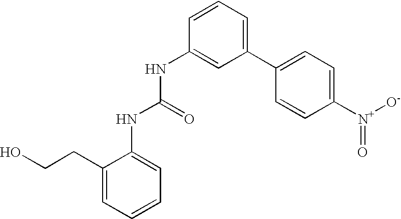 | 377,40328 | | 197-198 | A |
| 230 | 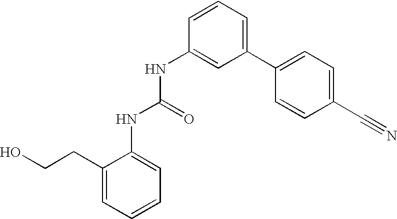 | 357,41563 | | 180-182 | A |
| 231 | 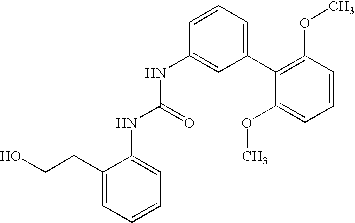 | 392,45873 | | 180-181 | C |
| 232 | 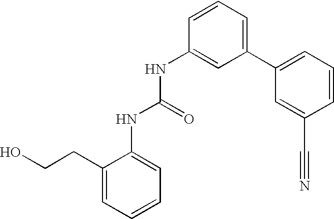 | 357,41563 | | 186-187 | A |
| 233 | 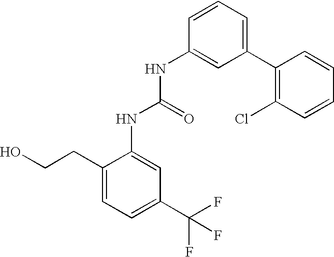 | 434,84916 | | 185-187 | B |
| 234 | 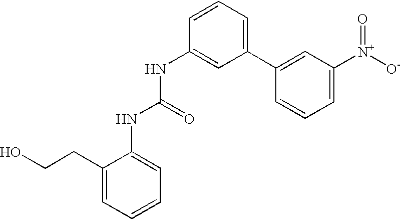 | 377,40328 | | 188 | A |
| 235 | 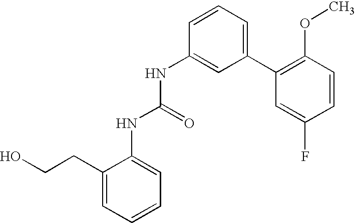 | 380,42267 | | 158-160 | A |
| 236 | 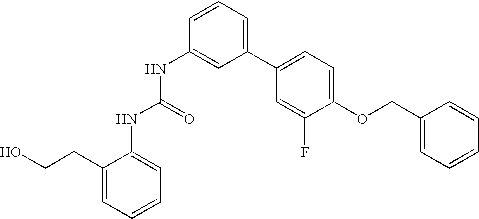 | 456,52145 | | 181-182 | A |
| 237 | 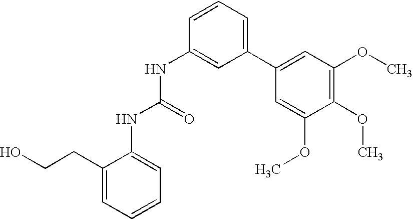 | 422,48522 | | 81-83 | B |
| 238 | 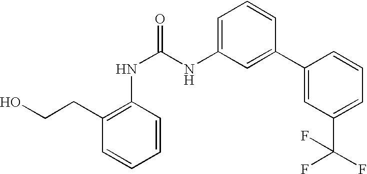 | 400,40413 | | 180 | A |
| 239 | 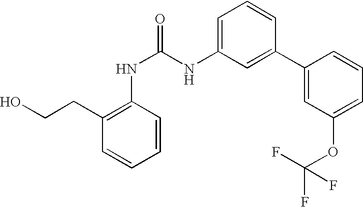 | 416,40353 | | 166 | A |
| 240 | 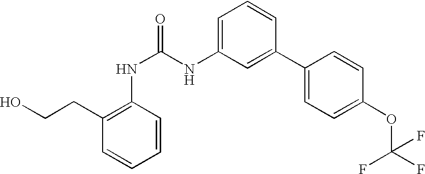 | 416,40353 | | 184 | A |
| 241 | 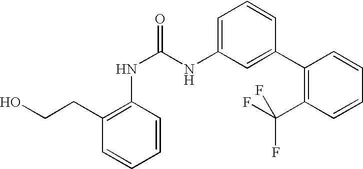 | 400,40413 | | 153 | A |
| 242 | 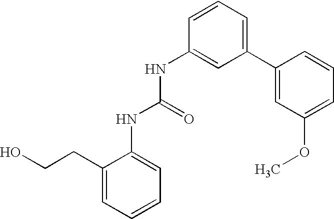 | 362,43224 | | 163 | A |
| 243 | 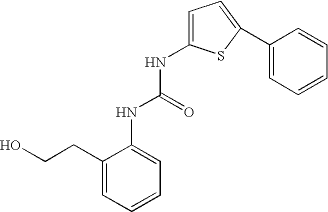 | 338,43151 | | 159 | A |
| 244 | 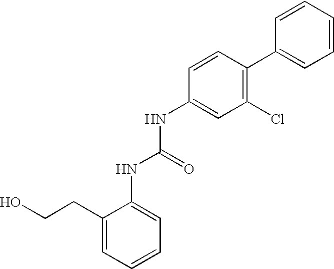 | 366,85078 | | 178 | B |
| 245 | 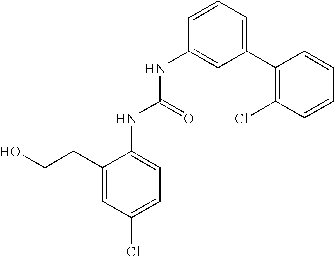 | 401,29581 | | 191-193 | B |
| 246 | 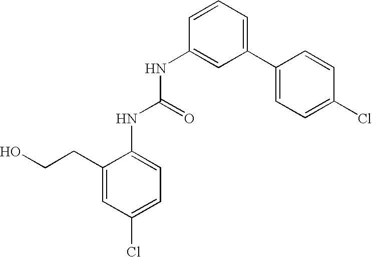 | 401,29581 | | 209-211 | C |
| 247 | 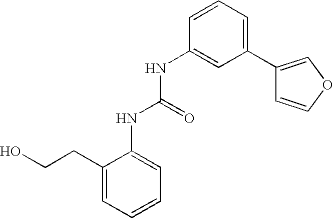 | 322,36691 | | 190 | A |
| 248 | 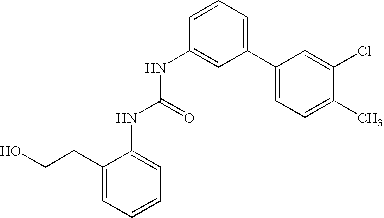 | 380,87787 | | 194 | A |
| 249 | 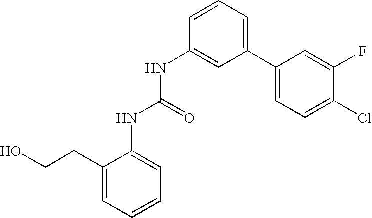 | 384,84121 | | 180 | A |
| 250 | 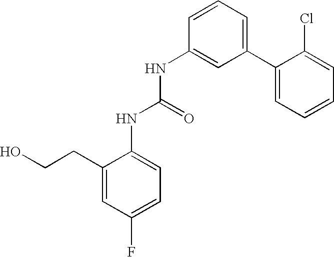 | 384,84121 | 369 | 177 | B |
| 251 | 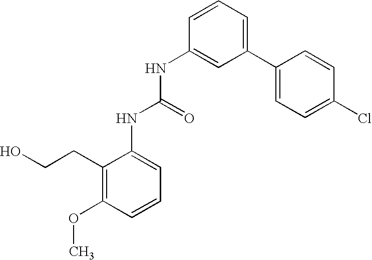 | 396,87727 | 385 | 195 | A |
| 252 | 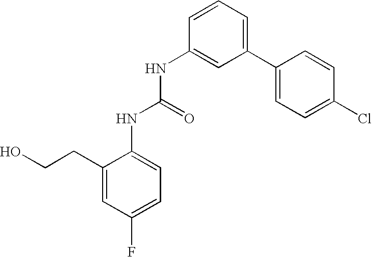 | 384,84121 | 397 | 187 | B |
| 253 | 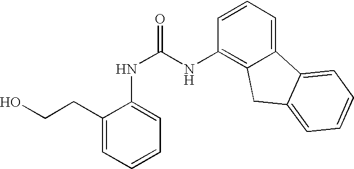 | 344,4169 | | 182-183 | A |
| 254 | 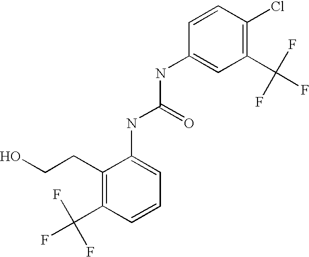 | 426,7488 | 427 | 201 | B |
| 255 | 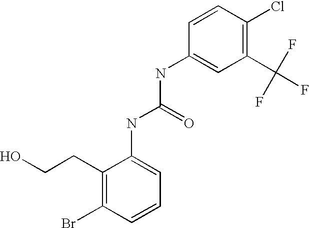 | 437,6464 | 438 | 209 | A |
| 256 | 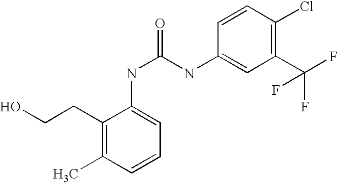 | 372,7775 | 373 | 201 | A |
| 257 | 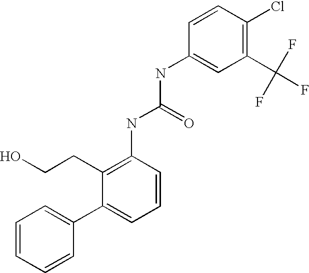 | 434,8492 | 435 | 173 | B |
| 258 | 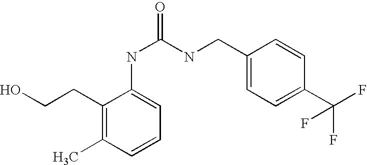 | 352,3595 | 353 | 213 | A |

1) an urea derivative of the formula (I), its tautomeric or stereoisomeric form, or a salt thereof: 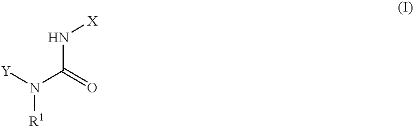
wherein
Y is 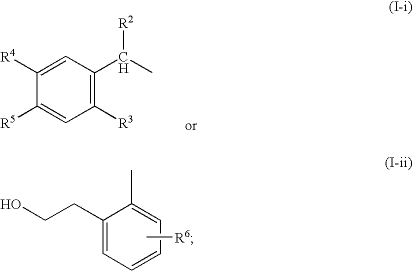
X is C₁₋₆ alkyl substituted by phenyl or naphthyl (wherein said phenyl and naphthyl are optionally substituted by R¹¹, R¹² and R¹³), aryl or heterocyclic ring,
wherein said aryl and heterocyclic ring are optionally substituted by R¹¹, R¹² and R¹³ and are are selected from the group consisting of phenyl, naphthyl, pyridyl, carbazolyl, fluorenyl, thienyl, pyrimidyl, benzodioxolyl, indazolyl, and quinolyl,
in which R¹¹, R¹² and R¹³ independently represent hydrogen, halogen, C₁₋₆ alkyl, mono-, di-, or tri-halogen substituted C₁₋₆ alkyl, nitro, cyano, C₁₋₆ alkoxy, hydroxy, piperidino, furyl, thienyl, benzyloxy, anilino, naphthyl, C₁₋₆ alkylcarbarmoyl, carbamoyl, carboxyl, amino, C₁₋₆ alkylamino, di(C₁₋₆ alkyl)amino, C₁₋₆ alkoxycarbonyl, benzyl, phenoxy, C₁₋₆ alkyl substituted phenoxy, pyridyl, halogen substituted phenoxy, C₁₋₆ alkylthio, C₁₋₆ alkanoyl, C₁₋₆ alkanoylamino, hydroxy substituted C₁₋₆ alkyl, mono-, di-, or tri-halogen substituted C₁₋₆ alkyloxy, or phenyl optionally substituted by one to three substituents,
in which the substituents are each different or identical and selected from the group consisting of hydrogen, halogen, C₁₋₆ alkyl, C₁₋₆ alkoxy, pyridyl, mono-, di-, or tri-halogen substituted C₁₋₆ alkyl, nitro, cyano, benzyloxy, thienyl, C₁₋₆alkanoyl, C₁₋₆ alkoxycarbonyl, C₁₋₆ alkylthio, di(C₁₋₆ alkyl)amino, and C₁₋₆ alkylamino, mono, di, or tri halogen substituted C₁₋₆ alkyloxy;
R¹ is hydrogen,
R² is hydrogen,
R³ is hydrogen, or
R² and R³ together form —(CH₂)ₘ— (wherein m represents 1, 2, 3 or 4), or
R¹ and R³ together form —(CH₂)ₙ— (wherein n represents 1, 2, or 3);
R⁴ is hydrogen, halogen, C₁₋₆ alkoxy, hydroxy, C₁₋₆ alkoxy substituted benzyloxy, sulfamoyl, C₁₋₆ alkylsulfamoyl, di(C₁₋₆ alkyl)sulfamoyl, di(C₁₋₆ alkyl)amino C₁₋₆ alkylene sulfamoyl, hydroxy C₁₋₆ alkyl piperazinosulfonyl, C₁₋₆ alkylsulfonylamino, nitro, amino, C₁₋₆ alkanoylamino, C₁₋₆ alkoxyC₁₋₆ alkyleneoxy,
R⁵ is hydrogen, halogen, C₁₋₆ alkoxy, hydroxy, C₁₋₆ alkoxy substituted benzyloxy, sulfamoyl, C₁₋₆ alkylsulfamoyl, di(C₁₋₆ alkyl)sulfamoyl, di(C₁₋₆ alkyl)amino C₁₋₆ alkylene sulfamoyl, hydroxy C₁₋₆ alkyl piperazinosulfonyl, C₁₋₆ alkylsulfonylamino, nitro, amino, C₁₋₆ alkanoylamino, C₁₋₆ alkoxyC₁₋₆ alkyleneoxy, or
R⁴ and R⁵ together form —O—(CH₂)—O—; and
R⁶ is hydrogen, halogen, C₁₋₆ alkyl, mono-, di-, or tri-halogen substituted C₁₋₆ alkyl, nitro, cyano, C₁₋₆ alkoxy, hydroxy, C₁₋₆ alkylcarbamoyl, carbamoyl, carboxyl, amino, C₁₋₆ alkylamino, di(C₁₋₆ alkyl)amino, C₁₋₆ alkoxycarbonyl, phenyl, benzyl, phenoxy, halogen substituted phenoxy, C₁₋₆ alkylthio, C₁₋₆ alkanoyl, C₁₋₆ alkanoylamino, hydroxy substituted C₁₋₄ alkyl, mono-, di-, or tri-halogen substituted C₁₋₆ alkoxy.

2) The urea derivative of the formula (I), its tautomeric or stereoisomeric form, or a salt thereof as claimed in claim 1,
wherein
Y is 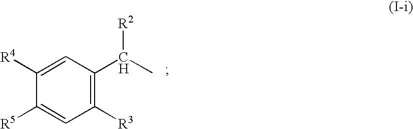
X is phenyl optionally substituted by R¹¹, R¹² and R¹³, phenyl C₁₋₆ alkyl (wherein said phenyl is optionally substituted by R¹¹, R¹² and R¹³), or naphthyl optionally substituted by R¹¹, R¹² and R¹³,
in which R¹¹, R¹² and R¹³ independently represent hydrogen, halogen, C₁₋₆ alkyl, mono-, di-, or tri-halogen substituted C₁₋₆ alkyl, nitro, C₁₋₆ alkoxy, C₁₋₆ alkoxycarbonyl, phenoxy, C₁₋₆ alkylthio, or C₁₋₆ alkanoyl.

3) The urea derivative of the formula (I), its tautomeric or stereoisomeric form, or a salt thereof as claimed in claim 1,
wherein
Y is 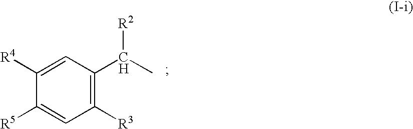
R¹ is hydrogen;
R² is hydrogen; and
R³ is hydrogen.

4) The urea derivative of the formula (I), its tautomeric or stereoisomeric form, or a salt thereof as claimed in claim 1,
wherein
Y is 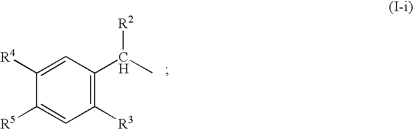
X is phenyl optionally substituted by R¹¹, R¹² and R¹³, phenyl C₁₋₆ alkyl (wherein said phenyl is optionally substituted by R¹¹, R¹² and R¹³), or naphthyl optionally substituted by R¹¹, R¹² and R¹³,
in which R¹¹, R¹² and R¹³ independently represent hydrogen, halogen, C₁₋₆ alkyl, mono-, di-, or tri-halogen substituted C₁₋₆ alkyl, nitro, C₁₋₆ alkoxy, C₁₋₆ alkoxycarbonyl, phenoxy, C₁₋₆ alkylthio, or Cab alkanoyl.
R¹ is hydrogen; and
R² and R³ together form —(CH₂)ₘ— (wherein m represents 1, 2, 3 or 4).

5) The urea derivative of the formula (I), its tautomeric or stereoisomeric form, or a salt thereof as claimed in claim 1,
wherein
Y is 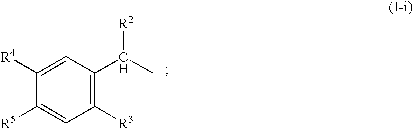
R¹ and R³ together form (CH₂)ₙ— (wherein n represents 1, 2, or 3); and
R² is hydrogen.

6) The urea derivative of the formula (I), its tautomeric or stereoisomeric form, or a salt thereof as claimed in claim 1,
wherein
Y is 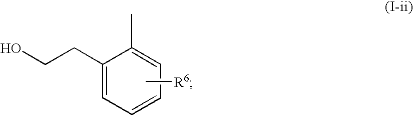
R⁶ is hydrogen, halogen, C₁₋₆ alkyl, mono-, di-, or tri-halogen substituted C₁₋₆ alkyl, phenyl or C₁₋₆ alkoxy.

7) The urea derivative of the formula (I), its tautomeric or stereoisomeric form, or a salt thereof as claimed in claim 1,
wherein
Y is 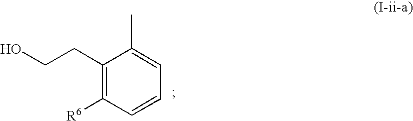
X is C₁₋₆ alkyl substituted by phenyl or naphthyl (wherein said phenyl and naphthyl are optionally substituted by R¹¹, R¹² and R¹³), aryl or heterocyclic ring,
wherein said aryl and heterocyclic ring are optionally substituted by R¹¹, R¹² and R¹³ and are selected from the group consisting of phenyl, naphthyl, pyridyl, carbazolyl, fluorenyl, thienyl, benzodioxolyl, indazolyl, and quinolyl,
in which R¹¹, R¹² and R¹³ independently represent hydrogen, halogen, C₁₋₆ alkyl, mono-, di-, or tri-halogen substituted C₁₋₆ alkyl, nitro, cyano, C₁₋₆ alkoxy, hydroxy, piperidino, furyl, thienyl, benzyloxy, anilino, naphthyl, di(C₁₋₆ alkyl)amino, C₁₋₆ alkoxycarbonyl, benzyl, phenoxy, C₁₋₆ alkyl substituted phenoxy, pyridyl, halogen substituted phenoxy, C₁₋₆ alkylthio, C₁₋₆ alkanoyl, C₁₋₆ alkanoylamino, hydroxy substituted C₁₋₆ alkyl, mono-, di-, or tri-halogen substituted C₁₋₆ alkyloxy,
or phenyl optionally substituted by one to three substituents, in which the substituents are each different or identical and selected from the group consisting of hydrogen, halogen, C₁₋₆ alkyl, C₁₋₆ alkoxy, pyridyl, mono-, di-, or tri-halogen substituted C₁₋₆ alkyl, nitro, cyano, benzyloxy, thienyl, C₁₋₆ alkanoyl, C₁₋₆ alkoxycarbonyl, C₁₋₆ alkylthio, di(C₁₋₆ alkyl)amino, C₁₋₄ alkylamino, and mono-, di- or tri-halogen substituted C₁₋₆ alkyloxy; and
R⁶ is hydrogen, halogen, C₁₋₆ alkyl, mono-, di-, or tri-halogen substituted C₁₋₆ alkyl, phenyl or C₁₋₆ alkoxy.

8) The urea derivative of the formula (I), its tautomeric or stereoisomeric form, or a salt thereof as claimed in claim 1, wherein said urea derivative of the formula (I) is selected from the group consisting of:
N-(4-hydroxy-3-methoxybenzyl)-N′-(4-isopropylphenyl)urea;
N-(4-hydroxy-3-methoxybenzyl)-N′-(1-naphthyl)urea;
N-(3,4-dichlorophenyl)-N′-(4-hydroxy-3-methoxybenzyl)urea;
N-(3-chloro-4-methylphenyl)-N′-(4-hydroxy-3 methoxybenzyl)urea;
N-(4-hydroxy-3-methoxybenzyl)-N′-(4-phenoxyphenyl)urea;
N-[2-chloro-5-(trifluoromethyl)phenyl]-N′-(4-hydroxy-3-methoxybenzyl)urea;
N-(3-chlorophenyl)-N′-(4-hydroxy-3-methoxybenzyl)urea;
N-(4-chlorophenyl)-N′-(4-hydroxy-3-methoxybenzyl)urea;
N-[4-chloro-3-(trifluoromethyl)phenyl]-N′-(4-hydroxy-3-methoxybenzyl)urea;
N-(4′-chloro-1,1′-biphenyl-3-yl)-N′-(4-hydroxy-3-methoxybenzyl)urea;
N-[2-(2-hydroxyethyl)phenyl]-N′-[4′-(methylsulfanyl)-1,1′-biphenyl-3-yl]urea;
N-[2-(2-hydroxyethyl)phenyl]-N′-(4′-nitro-1,1′-biphenyl-3-yl)urea;
N-(4′-acetyl-1,1′-biphenyl-3-yl)-N′-[2-(2-hydroxyethyl)phenyl]urea;
Ethyl3′-[({[2-(2-hydroxyethyl)phenyl]amino}carbonyl)amino]-1,1′-biphenyl-4-carboxylate;
N-[2-(2-hydroxyethyl)phenyl]-N′-[2′-(trifluoromethyl)-1,1′-biphenyl-3-yl]urea;
N-(2′-chloro-1,1′-biphenyl-3-yl)-N′-[2-(2-hydroxyethyl)phenyl]urea;
N-[2-(2-hydroxyethyl)phenyl]-N′-[3-(1-naphthyl)phenyl]urea;
N-[2-(2-hydroxyethyl)phenyl]-N′-[4′-(trifluoromethyl)-1,1′-biphenyl-3-yl]urea;
N-(4′,6-dichloro-1,1′-biphenyl-3-yl)-N′-[2-(2-hydroxyethyl)phenyl]urea;
N-(2′,5′-dichloro-1,1′-biphenyl-3-yl)-N′-[2-(2-hydroxyethyl)phenyl]urea;
N-(2′,4′-dichloro-1,1′-biphenyl-3-yl)-N′-[2-(2-hydroxyethyl)phenyl]urea;
N-(3′,4′-difluoro-1,1′-biphenyl-3-yl)-N′-[2-(2-hydroxyethyl)phenyl]urea;
N-(4′-fluoro-1,1′-biphenyl-3-yl)-N′-[2-(2-hydroxyethyl)phenyl]urea;
N-[2-(2-hydroxyethyl)phenyl]-N′-(3′-nitro-1,1′-biphenyl-3-yl)urea;
N-[4′-(benzyloxy)-3′-fluoro-1,1′-biphenyl-3-yl]-N′-[2-(2-hydroxyethyl)phenyl]urea;
N-(4′-chloro-1,1′-biphenyl-3-yl)-N′-[2-(2-hydroxyethyl)phenyl]urea;
N-(2′,5′-dimethyl-1,1′-biphenyl-3-yl)-N′-[2-(2-hydroxyethyl)phenyl]urea;
N-[2-(2-hydroxyethyl)phenyl]-N′-[4′-(trifluoromethoxy)-1,1′-biphenyl-3-yl]urea;
N-(4′-chloro-1,1′-biphenyl-3-yl)-N′-[2-(2-hydroxyethyl)-3-methoxyphenyl]urea;
N-(3′-fluoro-1,1′-biphenyl-3-yl)-N′-[2-(2-hydroxyethyl)phenyl]urea;
N-(3′-chloro-1,1′-biphenyl-3-yl)-N′-[2-(2-hydroxyethyl)phenyl]urea;
N-(2′,5′-difluoro-1,1′-biphenyl-3-yl)-N′-[2-(2-hydroxyethyl)phenyl]urea; and
N-(3′-chloro-4′-fluoro-1,1′-biphenyl-3-yl)-N′-[2-(2-hydroxyethyl)phenyl]urea.

9) An urea derivative of the formula (I), its tautomeric or stereoisomeric form, or a salt thereof as claimed in claims 1 for the treatment and/or prophylaxis of diseases.

10) A medicament comprising the urea derivative, its tautomeric or stereoisomeric form, or a physiologically acceptable salt thereof as claimed in claim 1 as an active ingredient.

11) The medicament as claimed in claim 10, further comprising one or more pharmaceutically acceptable excipients.

12). The medicament as claimed in claim 10, wherein the urea derivative, its tautomeric or stereoisomeric form, or a physiologically acceptable salt thereof is a VR1 antagonist.

13) The medicament as claimed in claim 10 for treatment and/or prophylaxis of a disease selected from the group consisting of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders.

14) An agent to treat or prevent urological disorder; comprising the urea derivative, its tautomeric or stereoisomeric form, or a physiologically acceptable salt thereof as claimed in claim 1 as an active ingredient.

15) An agent to treat or prevent of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders; comprising the urea derivative, its tautomeric or stereoisomeric form, or a physiologically acceptable salt thereof as claimed in claim 1 as an active ingredient.

16) A method for treating or preventing disorder or disease associated with VR1 activity in a human or animal subject, comprising administering to said subject a therapeutically effective amount of the urea derivative, its tautomeric or stereoisomeric form, or a physiologically acceptable salt thereof as claimed in claim 1.

17) The method of claim 16, wherein said disorder or disease is a urological disorder or disease.

18) The method of claim 16, wherein said disorder or disease is selected from the group consisting of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders.

19) The method of claim 16, wherein said urea derivative, its tautomeric or stereoisomeric form, or a physiologically acceptable salt thereof is administered with one or more pharmaceutically acceptable excipients.

20) Use of the urea derivative, its tautomeric or stereoisomeric form, or a physiologically acceptable salt thereof as claimed in claim 1 in the preparation of a medicament.

21) Use of urea derivative, its tautomeric or stereoisomeric form, or a physiologically acceptable salt thereof as claimed in claim 1 in the preparation of a medicament for treating or preventing disorder or disease associated with VR1 activity.

22) The use of claim 21, wherein said disorder or disease is urological disorder or disease.

23) The use of claim 21, wherein said disorder or disease is selected from the group consisting of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders.

24) The use of claim 21, wherein said urea derivative, its tautomeric or stereoisomeric form, or a physiologically acceptable salt thereof is formulated with one or more pharmaceutically acceptable excipients.

25) Process for controlling urological disorders in humans and animals by administrating of a VR1 antagonisticly effective amount of at least one compound as claimed in claim 1.
